(19) United States
(12) Patent Application Publication
(10) Pub. No.: US 2024/0207465 A1
(43) Pub. Date: Jun. 27, 2024

(54) VIBRATION APPARATUS AND APPARATUS INCLUDING THE SAME

(71) Applicant: LG DISPLAY CO., LTD., SEOUL (KR)

(72) Inventors: Sooyoun Kim, Gyeonggi-do (KR); YongWoo Lee, Gyeonggi-do (KR); JounHo Lee, Gyeonggi-do (KR); HwaYoul Lee, Gyeonggi-do (KR); Uihyeon Jeong, Gyeonggi-do (KR)

(73) Assignee: LG DISPLAY CO., LTD., SEOUL (KR)

(21) Appl. No.: 18/383,670

(22) Filed: Oct. 25, 2023

(30) Foreign Application Priority Data
Dec. 27, 2022 (KR) 10-2022-0186138

(51) Int. Cl. A61L 2/025; A61L 2/26; C04B 35/495; G06F 3/01; H10N 39/00

(52) U.S. Cl. CPC A61L 2/025; A61L 2/26; C04B 35/495; G06F 3/016; H10N 39/00; C04B 2235/3255; H10N 30/8542

(57) ABSTRACT

A vibration apparatus includes a base member, a protection member, and a vibration generating part configured between the base member and the protection member to output an ultrasound, wherein the vibration generating part includes a plurality of first signal lines and a plurality of second signal lines configured on the base member to intersect with one another and a plurality of vibration parts disposed at intersections of the plurality of first signal lines and the plurality of second signal lines.

### CROSS-REFERENCE TO RELATED APPLICATIONS

This application claims the benefit of and priority to Korean Patent Application No. 10-2022-0186138 filed on Dec. 27, 2022, the entirety of which is hereby incorporated by reference for all purposes as if fully set forth herein.

### TECHNICAL FIELD

The present disclosure relates to a vibration apparatus and an apparatus including the same, and more particularly, to a vibration apparatus for generating an ultrasound and an apparatus including the same.

### BACKGROUND

As information-oriented society advances, the needs for display apparatuses for displaying an image are variously increasing.

Apparatuses (or electronic devices) using a display apparatus as a display screen provide a user interface of a touch screen type, for convenience of a user input.

Screens of apparatuses (or electronic devices) such as automated teller machines or kiosks using a touch screen type may be exposed at an external environment or unauthorized users, and due to this, may be polluted by harmful bacteria such as various kinds of bacteria and/or viruses from an external environment or a user.

In ultraviolet disinfection (or sterilization) a method of disinfecting bacteria and/or virus. When there is no user, sterilization starts in an ultraviolet lamp, and when a user approaches the ultraviolet lamp, a proximity sensor operates to stop sterilization. However, when the proximity sensor operates abnormally, the user is exposed to ultraviolet rays.

In a method of sterilizing bacteria and/or virus, ion sterilization may use copper (Cu) or silver (Ag). However, there is a limitation in forming all portions, which a hand of a user can be proximate to, with ions of copper. Therefore, there is a problem where an antimicrobial film when a copper ion is added to polyethylene terephthalate or a polyethylene film is used but a sterilization time is short.

In another method, ultrasound cleaning is used, a vibrator used in ultrasound cleaning includes lead (Pb), and a weight ratio of lead (Pb) included in the vibrator is about 60% or more, causing a problem of environmental pollution.

Harmful bacteria of a screen of an apparatus are capable of being propagated to a user when the user touches a screen which can spread the bacteria.

### SUMMARY

Various research and experiments for providing a vibration apparatus and an apparatus including the same are disclosed. The apparatus may sterilize or disinfect harmful bacteria of a surface of a screen. Based on the various research and experiments, the disclosure provides an apparatus which may generate an ultrasound for surface sterilization and/or surface disinfection of a screen.

An aspect of the present disclosure is directed to providing a vibration apparatus and an apparatus including the same, which may generate an ultrasound for surface sterilization and/or surface disinfection of a screen.

Another aspect of the present disclosure is directed to providing a vibration apparatus and an apparatus including the same, which may generate an ultrasound for removing foreign materials such as dust.

Another aspect of the present disclosure is directed to providing a vibration apparatus and an apparatus including the same, which may generate an ultrasound for surface sterilization and/or surface disinfection of a screen by using a Pb-free piezoelectric material.

Additional features, advantages, and aspects of the present disclosure are set forth in the present disclosure and will also be apparent from the present disclosure or may be learned by practice of the inventive concepts provided herein. Other features, advantages, and aspects of the present disclosure may be realized and attained by the structure particularly pointed out in the present disclosure, or derivable therefrom, and claims hereof as well as the appended drawings.

To achieve these and other advantages and aspects of the present disclosure, as embodied and broadly described herein, in one or more aspects, a vibration apparatus may comprise a base member, a protection member, and a vibration generating part configured between the base member and the protection member to output an ultrasound. The vibration generating part may comprise a plurality of first signal lines and a plurality of second signal lines configured on the base member to intersect with one another, and a plurality of vibration parts disposed at intersections of the plurality of first signal lines and the plurality of second signal lines.

In another aspect of the present disclosure, an apparatus may comprise a front member, and a vibration generating apparatus configured to generate an ultrasound at a surface of a front member. The vibration generating apparatus may comprise a vibration apparatus. The vibration apparatus may comprise a base member, a protection member, and a vibration generating part configured between the base member and the protection member to output an ultrasound. The vibration generating part may comprise a plurality of first signal lines and a plurality of second signal lines configured on the base member to intersect with one another, and a plurality of vibration parts disposed at intersections of the plurality of first signal lines and the plurality of second signal lines.

In another aspect of the present disclosure, an apparatus may comprise a display apparatus at a rear surface of a front member, and a vibration generating apparatus between the front member and the display apparatus to vibrate the front member and generate an ultrasound at a surface of the front member. The vibration generating apparatus may comprise a vibration apparatus. The vibration apparatus may comprise a base member, a protection member, and a vibration generating part between the base member and the protection member to output an ultrasound. The vibration generating part may comprise a plurality of first signal lines and a plurality of second signal lines configured on the base member to intersect with one another, and a plurality of vibration parts disposed at intersection parts between the plurality of first signal lines and the plurality of second signal lines.

A vibration apparatus and an apparatus including the same according to one or more aspects of the present disclosure may generate an ultrasound for surface sterilization and/or surface disinfection of a screen.

A vibration apparatus and an apparatus including the same according to one or more aspects of the present disclosure may generate an ultrasound for removing foreign materials such as dust.

A vibration apparatus and an apparatus including the same according to one or more aspects of the present disclosure may generate an ultrasound for surface sterilization and/or surface disinfection of a screen by using a Pb-free piezoelectric material.

A vibration apparatus and an apparatus including the same according to one or more aspects of the present disclosure may use a Pb-free piezoelectric material, and thus, may prevent environmental pollution caused by toxicity of lead (Pb) and a harmful material occurring in a sintering process, reduce production harmfulness/regulation material, provide an environment-friendly product, and replace a harmful material.

Other systems, methods, features and advantages will be, or will become, apparent to one with skill in the art upon examination of the following figures and detailed description. It is intended that all such additional systems, methods, features and advantages be included within this description, be within the scope of the present disclosure, and be protected by the following claims. Nothing in this section should be taken as a limitation on those claims. Further aspects and advantages are discussed below in conjunction with aspects of the disclosure.

### BRIEF DESCRIPTION OF THE DRAWINGS

The accompanying drawings, which are included to provide a further understanding of the disclosure and are incorporated in and constitute a part of this disclosure, illustrate aspects of the disclosure and together with the description serve to explain principles of the disclosure.

FIG. 1 illustrates a vibration apparatus according to an aspect of the present disclosure.

FIG. 2 is a cross-sectional view taken along line I-I′ illustrated in FIG. 1 according to an aspect of the present disclosure.

FIG. 3 illustrates a base member and a vibration generating apparatus illustrated in FIG. 1 according to an aspect of the present disclosure.

FIG. 4 illustrates a vibration apparatus according to another aspect of the present disclosure.

FIG. 5 illustrates an apparatus according to an aspect of the present disclosure.

FIG. 6 illustrates an apparatus according to another aspect of the present disclosure.

FIG. 7 illustrates a vibration generating apparatus and a touch panel illustrated in FIG. 6 according to another aspect of the present disclosure.

FIG. 8 illustrates an apparatus according to another aspect of the present disclosure.

FIG. 9 illustrates a front member and a vibration generating apparatus illustrated in FIG. 8 according to another aspect of the present disclosure.

Throughout the drawings and the detailed description, unless otherwise described, the same drawing reference numerals should be understood to refer to the same elements, features, and structures. The sizes, lengths, and thicknesses of layers, regions and elements, and depiction thereof may be exaggerated for clarity, illustration, and/or convenience.

### DETAILED DESCRIPTION

Reference is now made in detail to aspects of the present disclosure, examples of which may be illustrated in the accompanying drawings. In the following description, when a detailed description of well-known functions, structures or configurations may unnecessarily obscure aspects of the present disclosure, a detailed description of such known functions or configurations may have been omitted for brevity. Further, repetitive descriptions may be omitted for brevity. The progression of processing steps and/or operations described is a non-limiting example.

The sequence of steps and/or operations is not limited to that set forth herein and may be changed to occur in an order that is different from an order described herein, with the exception of steps and/or operations necessarily occurring in a particular order. In one or more examples, two operations in succession may be performed substantially concurrently, or the two operations may be performed in a reverse order or in a different order depending on a function or operation involved.

Unless stated otherwise, like reference numerals may refer to like elements throughout even when they are shown in different drawings. In one or more aspects, identical elements (or elements with identical names) in different drawings may have the same or substantially the same functions and properties unless stated otherwise. Names of the respective elements used in the following explanations are selected only for convenience and may be thus different from those used in actual products.

Advantages and features of the present disclosure, and implementation methods thereof, are clarified through the following example aspects described with reference to the accompanying drawings. The present disclosure may, however, be embodied in different forms and should not be construed as limited to the example aspects set forth herein. Rather, these example aspects are provided so that this disclosure may be sufficiently thorough and complete to assist those skilled in the art to understand the inventive concepts fully without limiting the protected scope of the present disclosure.

Shapes (e.g., sizes, lengths, widths, heights, thicknesses, locations, radii, diameters, and areas), dimensions, ratios, angles, numbers, and the like disclosed herein, including those illustrated in the drawings are merely examples. Any implementation described herein as an “example” is not necessarily to be construed as preferred or advantageous over other implementations. It is, however, noted that the relative dimensions of the components illustrated in the drawings are part of the present disclosure.

When a term like “comprise,” “have,” “include,” “contain,” “constitute,” “made of,” or “formed of,” or the like is used with respect to one or more elements, one or more other elements may be added unless a more limiting term, such as “only” or the like, is used. The terms used in the present disclosure are merely used in order to describe particular aspects and are not intended to limit the scope of the present disclosure. The terms of a singular form may include plural forms unless the context clearly indicates otherwise.

The word “exemplary” is used to mean serving as an example or illustration, unless otherwise specified. “Embodiments,” “examples,” “aspects,” and the like should not be construed as preferred or advantageous over other implementations. An embodiment, an example, an example embodiment, an aspect, or the like may refer to one or more embodiments, one or more examples, one or more example embodiments, one or more aspects, or the like, unless stated otherwise. Further, the term “may” encompasses all the meanings of the term “can.”

In one or more aspects, unless explicitly stated otherwise, an element, feature, or corresponding information (e.g., a level, range, dimension, size, or the like) is construed to include an error or tolerance range even where no explicit description of such an error or tolerance range is provided. An error or tolerance range may be caused by various factors (e.g., process factors, internal or external impact, noise, or the like). In interpreting a numerical value, the value is interpreted as including an error range unless explicitly stated otherwise.

In describing a positional relationship when the positional relationship between two parts (e.g., layers, films, regions, components, sections, or the like) is described, for example, using “on,” “upon,” “on top of.” “over,” “under,” “above,” “below,” “beneath,” “near,” “close to,” “adjacent to,” “beside,” “next to,” “at or on a side of,” or the like, one or more other parts may be located between the two parts unless a more limiting term, such as “immediate(ly),” “direct(ly),” or “close(ly),” is used. For example, where a structure is described as being positioned “on,” “on a top of,” “upon,” “on top of,” “over,” “under,” “above,” “below,” “beneath,” “near,” “close to,” “adjacent to,” “beside,” “next to,” “at or on a side of,” or the like another structure, this description should be construed as including a case in which the structures contact each other as well as a case in which one or more additional structures are disposed therebetween. Furthermore, the terms “front,” “rear,” “back,” “left,” “right,” “top,” “bottom,” “downward,” “upward,” “upper,” “lower,” “up,” “down,” “column,” “row;” “vertical,” “horizontal,” and the like refer to an arbitrary frame of reference, unless otherwise specified.

Spatially relative terms, such as “below,” “beneath,” “lower,” “on,” “above,” “upper” and the like, can be used to describe a correlation between various elements (e.g., layers, films, regions, components, sections, or the like) as shown in the drawings. The spatially relative terms are to be understood as terms including different orientations of the elements in use or in operation in addition to the orientation depicted in the drawings. For example, if the elements shown in the drawings are turned over, elements described as “below” or “beneath” other elements would be oriented “above” other elements. Thus, the term “below,” which is an example term, can include all directions of “above” and “below.” Likewise, an exemplary term “above” or “on” can include both directions of “above” and “below.”

In describing a temporal relationship, when the temporal order is described as, for example, “after,” “subsequent,” “next,” “before,” “preceding,” “prior to,” or the like, a case that is not consecutive or not sequential may be included and thus one or more other events may occur therebetween, unless a more limiting term, such as “just,” “immediate(ly),” or “direct(ly),” is used.

The terms, such as “below,” “lower,” “above,” “upper” and the like, may be used herein to describe a relationship between element(s) as illustrated in the drawings. It will be understood that the terms are spatially relative and based on the orientation depicted in the drawings.

It is understood that, although the terms “first,” “second,” or the like may be used herein to describe various elements (e.g., layers, films, regions, components, sections, or the like), these elements should not be limited by these terms, for example, to any particular order, precedence, or number of elements. These terms are used only to distinguish one element from another. For example, a first element could be termed a second element, and, similarly, a second element could be termed a first element, without departing from the scope of the present disclosure. Furthermore, the first element, the second element, and the like may be arbitrarily named according to the convenience of those skilled in the art without departing from the scope of the present disclosure. For clarity, the functions or structures of these elements (e.g., the first element, the second element and the like) e not limited by ordinal numbers or the names in front of the elements. Further, a first element may include one or more first elements. Similarly, a second element or the like may include one or more second elements or the like.

In describing elements of the present disclosure, the terms “first,” “second.” “A,” “B.” “(a),” “(b),” or the like may be used. These terms are intended to identify the corresponding element(s) from the other element(s), and are not used to define the essence, basis, order, or number of the elements.

When an element (e.g., layer, film, region, component, section, or the like) is described as “connected.” “coupled.” “attached,” “adhered,” or the like to another element, the element may not only be directly connected, coupled, attached, adhered, or the like to another element, but also be indirectly connected, coupled, attached, adhered, or the like to another element with one or more intervening elements disposed or interposed between the elements, unless otherwise specified.

For the expression that an element (e.g., layer, film, region, component, section, or the like) “contacts,” “overlaps,” or the like with another element, the element or layer may not only directly contact, overlap, or the like with another element, but also indirectly contact, overlap, or the like with another element with one or more intervening elements disposed or interposed between the elements, unless otherwise specified.

The phase that an element (e.g., layer, film, region, component, section, or the like) is “provided in,” “disposed in,” or the like in another element may be understood as that at least a portion of the element is provided in, disposed in, or the like in another element, or that the entirety of the element is provided in, disposed in, or the like in another element. The phase that an element (e.g., layer, film, region, component, section, or the like) “contacts,” “overlaps,” or the like with another element may be understood as that at least a portion of the element contacts, overlaps, or the like with a least a portion of another element, that the entirety of the element contacts, overlaps, or the like with a least a portion of another element, or that at least a portion of the element contacts, overlaps, or the like with the entirety of another element.

Such terms as a “line” or “direction” should not be interpreted only based on a geometrical relationship in which the respective lines or directions are parallel or perpendicular to each other. Such terms may mean a wider range of lines or directions within which the components of the present disclosure may operate functionally. For example, the terms “first direction,” “second direction,” and the like, such as a direction parallel or perpendicular to “x-axis,” “y-axis,” or “z-axis,” should not be interpreted only based on a geometrical relationship in which the respective directions are parallel or perpendicular to each other, and may be meant as directions having wider directivities within the range within which the components of the present disclosure can operate functionally.

The term “at least one” should be understood as including any and all combinations of one or more of the associated listed items. For example, each of the phrases of “at least one of a first item, a second item, or a third item” and “at least one of a first item, a second item, and a third item” may represent (i) a combination of items provided by two or more of the first item, the second item, and the third item or (ii) only one of the first item, the second item, or the third item.

The expression of a first element, a second elements, “and/or” a third element should be understood to encompass one of the first, second, and third elements, as well as any and all combinations of the first, second and third elements. By way of example, A, B and/or C encompass only A; only B; only C; any of A, B, and C (e.g., A, B, or C); or some combination of A, B, and C (e.g., A and B; A and C; or B and C); or all of A, B, and C. Furthermore, an expression “A/B” may be understood as A and/or B. For example, an expression “A/B” can refer to only A; only B; A or B; or A and B.

In one or more aspects, the terms “between” and “among” may be used interchangeably simply for convenience unless stated otherwise. For example, an expression “between a plurality of elements” may be understood as among a plurality of elements. In another example, an expression “among a plurality of elements” may be understood as between a plurality of elements. In one or more examples, the number of elements may be two. In one or more examples, the number of elements may be more than two. Furthermore, when an element (e.g., layer, film, region, component, sections, or the like) is referred to as being “between” at least two elements, the element may be the only element between the at least two elements, or one or more intervening elements may also be present.

In one or more aspects, the phrases “each other” and “one another” may be used interchangeably simply for convenience unless stated otherwise. For example, an expression “different from each other” may be understood as different from one another. In another example, an expression “different from one another” may be understood as different from each other. In one or more examples, the number of elements involved in the foregoing expression may be two. In one or more examples, the number of elements involved in the foregoing expression may be more than two.

In one or more aspects, the phrases “one or more among” and “one or more of” may be used interchangeably simply for convenience unless stated otherwise.

The term “or” means “inclusive or” rather than “exclusive or.” That is, unless otherwise stated or clear from the context, the expression that “x uses a or b” means any one of natural inclusive permutations. For example, “a or b” may mean “a,” “b,” or “a and b.” For example, “a, b or c” may mean “a,” “b,” “c,” “a and b,” “b and c,” “a and c,” or “a, b and c.”

Features of various aspects of the present disclosure may be partially or entirety coupled to or combined with each other, may be technically associated with each other, and may be operated, linked or driven together in various ways. Aspects of the present disclosure may be implemented or carried out independently of each other, or may be implemented or carried out together in a co-dependent or related relationship. In one or more aspects, the components of each apparatus may be operatively coupled and configured.

Unless otherwise defined, the terms (including technical and scientific terms) used herein have the same meaning as commonly understood by one of ordinary skill in the art to which example aspects belong. It should be further understood that terms, such as those defined in commonly used dictionaries, should be interpreted as having a meaning that is, for example, consistent with their meaning in the context of the relevant art and should not be interpreted in an idealized or overly formal sense, unless expressly defined otherwise herein.

The terms used herein have been selected as being general in the related technical field: however, there may be other terms depending on the development and/or change of technology, convention, preference of technicians, and so on. Therefore, the terms used herein should not be understood as limiting technical ideas, but should be understood as examples of the terms for describing example embodiments.

Further, in a specific case, a term may be arbitrarily selected by an applicant, and in this case, the detailed meaning thereof is described herein. Therefore, the terms used herein should be understood based on not only the name of the terms, but also the meaning of the terms and the content hereof.

In the following description, various example aspects of the present disclosure are described in detail with reference to the accompanying drawings. With respect to reference numerals to elements of each of the drawings, the same elements may be illustrated in other drawings, and like reference numerals may refer to like elements unless stated otherwise. The same or similar elements may be denoted by the same reference numerals even though they are depicted in different drawings. In addition, for convenience of description, a scale, dimension, size, and thickness of each of the elements illustrated in the accompanying drawings may be different from an actual scale, dimension, size, and thickness. Thus, aspects of the present disclosure are not limited to a scale, dimension, size, or thickness illustrated in the drawings.

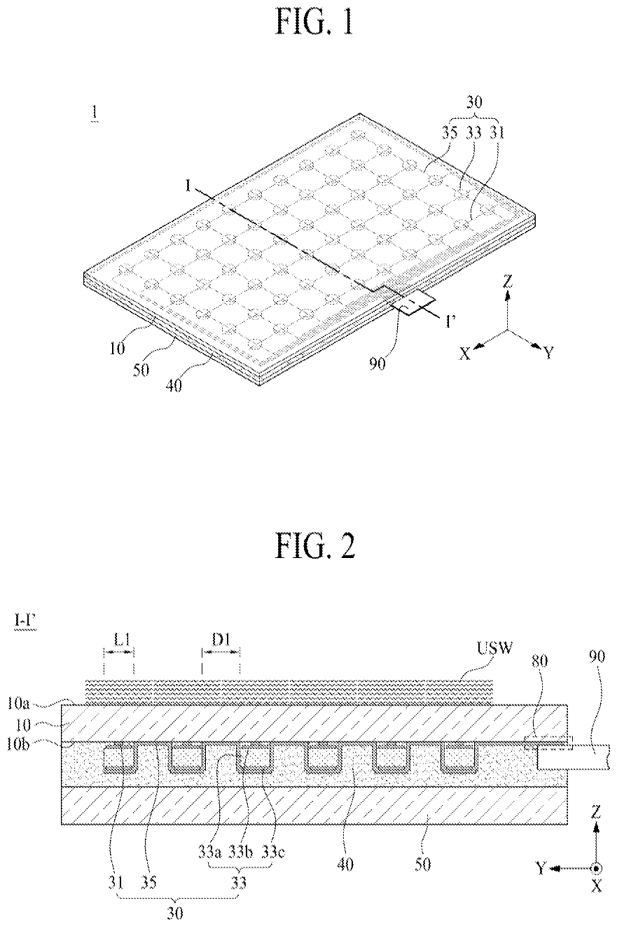

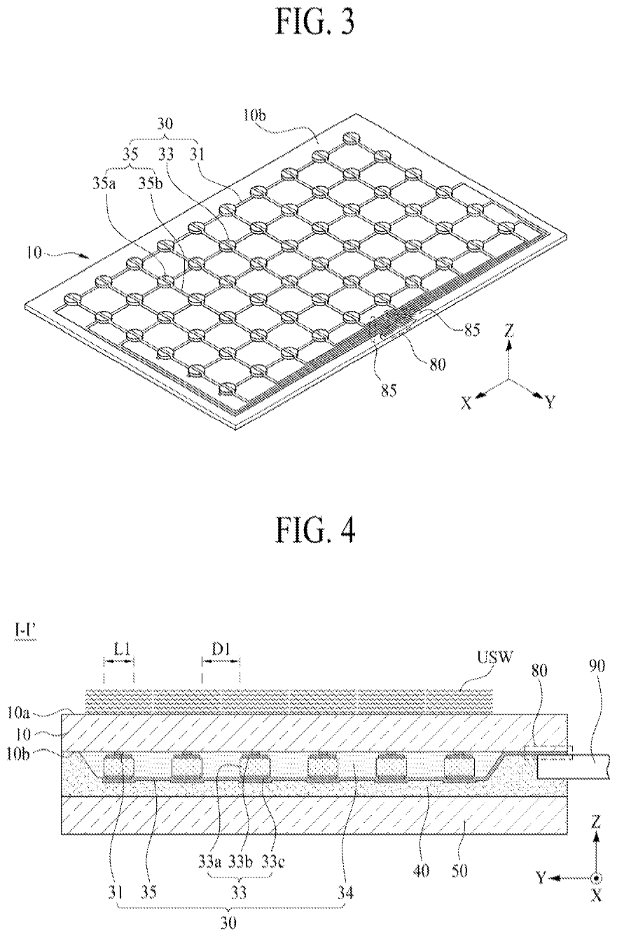

FIG. 1 illustrates a vibration apparatus according to an aspect of the present disclosure. FIG. 2 is a cross-sectional view taken along line I-I′ illustrated in FIG. 1 according to an aspect of the present disclosure. FIG. 3 illustrates a base member and a vibration generating apparatus illustrated in FIG. 1 according to an aspect of the present disclosure.

Referring to FIGS. 1 and 2, a vibration apparatus 1 according to an aspect of the present disclosure may include a base member 10, a vibration generating part 30, and a protection member 50.

The base member 10 may comprise a base substrate, a lower substrate, or a base film, or the like, but aspects of the present disclosure are not limited thereto. The base member 10 may be configured as a transparent plastic material or a transparent glass material. For example, the base member 10 may be configured as one of polyimide, polyethylene terephthalate, polyurethane, cyclo-olefin polymer, and triacetylcellulose, or a combination material thereof, but aspects of the present disclosure are not limited thereto.

The base member 10 may include a first surface 10a and a second surface 10b opposite to the first surface 10a. The first surface 10a of the base member 10 may be a front surface, an external surface, or an outer surface. The second surface 10b of the base member 10 may be a rear surface, a backside surface, an internal surface, or an inner surface.

The vibration generating part 30 may be configured at the base member 10. The vibration generating part 30 may be configured at the base member 10 to vibrate based on a driving signal (e.g., a vibration signal or an ultrasound driving signal) applied from the outside to generate (or output) an ultrasound USW. The vibration generating part 30 may vibrate the base member 10 based on the driving signal to output (or generate) the ultrasound USW. The vibration generating part 30 may be configured at the second surface 10b of the base member 10. For example, the ultrasound USW generated (or output) by the vibration generating part 30 may have a frequency of 25 MHz or more. For example, the ultrasound USW generated by the vibration generating part 30 may have a frequency of 100 MHz to 350 MHZ, but aspects of the present disclosure are not limited thereto. The vibration generating part 30 may also be referred to as a sound wave generating part, a sound wave generating device, a sound wave vibration part, a sound wave vibrator, a sound wave piezoelectric device, an ultrasonic generating part, an ultrasonic generating device, an ultrasonic vibration part, an ultrasonic vibrator, or an ultrasound piezoelectric device, or the like, but aspects of the present disclosure are not limited thereto.

The vibration generating part 30 according to an aspect of the present disclosure may include a plurality of first signal lines 31, a plurality of vibration parts 33, and a plurality of second signal lines 35. For example, the vibration generating part 30 may include a plurality of first signal lines 31 and a plurality of second signal lines 35, which are arranged on the base member 10 to intersect with one another, and a plurality of vibration parts 33 which are disposed or interposed between the plurality of first signal lines 31 and the plurality of second signal lines 35 in intersection parts (or intersection regions) between the plurality of first signal lines 31 and the plurality of second signal lines 35.

The plurality of first signal lines 31 may be disposed on the second surface 10b of the base member 10. For example, the plurality of first signal lines 31 may extend along a first direction X and may be separated from one another along a second direction Y and intersecting the first direction X at the second surface 10b of the base member 10. For example, the first direction X may be a long-side lengthwise direction, a widthwise direction, or a horizontal direction of the vibration apparatus or the base member 10, or an X-axis direction in an XYZ-coordinate system. For example, the second direction Y may be a short-side lengthwise direction, a lengthwise direction, or a vertical direction of the vibration apparatus or the base member 10, or a Y-axis direction in an XYZ-coordinate system.

The plurality of first signal lines 31 may contact the base member 10. For example, the plurality of first signal lines 31 may directly contact the base member 10. The plurality of first signal lines 31 may be formed directly at the second surface 10b of the base member 10. The plurality of first signal lines 31 may be formed at the second surface 10b of the base member 10 by patterning a transparent conductive layer (e.g., deposited or formed) on an entire second surface 10b of the base member 10, but aspects of the present disclosure are not limited thereto. The plurality of first signal lines 31 may be formed by a sputtering process using a mask.

The plurality of vibration parts 33 may be disposed on the second surface 10b of the base member 10 to be electrically connected to the plurality of first signal lines 31. For example, the plurality of vibration parts 33 may be configured or disposed on (or over) the second surface 10b of the base member 10 to be electrically connected to the plurality of first signal lines 31. The plurality of vibration parts 33 may be configured or disposed to have an arrangement structure having a lattice pattern shape. For example, the plurality of vibration parts 33 may be separated from one another in each of the first direction X and the second direction Y and may be electrically connected to a corresponding first signal line of the plurality of first signal lines 31.

Each of the plurality of vibration parts 33 may include a circular plate shape, a polygonal plate shape, an oval shape, a disc shape, or a ring shape, but aspects of the present disclosure are not limited thereto. A separation interval DI between the plurality of vibration parts 33 may be 0.5 cm to 8 cm. For example, the separation interval DI between the plurality of vibration parts 33 along each of the first direction X and the second direction Y may be 0.5 cm to 8 cm. For example, when the separation interval DI between the plurality of vibration parts 33 is less than 0.5 cm, vibration interference between the plurality of vibration parts 33 may occur due to smaller pitch, which is related to the separation interval based on a size of the vibration parts. When the separation interval DI between the plurality of vibration parts 33 is greater than 8 cm, a dead zone between the plurality of vibration parts 33 may be formed. The ultrasound USW is not output (or does not occur) in the dead zone due to a wide interval.

Each of the plurality of vibration parts 33 may include a piezoelectric layer 33a, a first electrode layer 33b, and a second electrode layer 33c.

The piezoelectric layer 33a may include a piezoelectric material having a piezoelectric effect (or a piezoelectric characteristic). For example, the piezoelectric layer 33a may be an inorganic layer, an inorganic material layer, a piezoelectric material layer, an electroactive layer, a piezoelectric portion, an inorganic portion, an inorganic material portion, a piezoelectric material portion, or an electroactive portion, or the like, but aspects of the present disclosure are not limited thereto.

In some aspects, the piezoelectric layer 33a may be configured as a ceramic-based material being capable of implementing a relatively strong vibration. In another aspect, the piezoelectric layer 33a may be configured as a piezoelectric ceramic having a perovskite-based crystalline structure. The perovskite crystalline structure may have a piezoelectric effect and/or an inverse piezoelectric effect and may be a plate-shaped structure having an orientation.

According to an aspect of the present disclosure, the piezoelectric layer 33a may include a lead zirconate titanate (PZT)-based piezoelectric material including lead (Pb), zirconium (Zr), and titanium (Ti) each having a high piezoelectric characteristic, but is not limited thereto. For example, the piezoelectric layer 33a may include a Pb-free piezoelectric material to prevent environmental pollution caused by toxicity of Pb and a harmful material occurring in a sintering process.

The piezoelectric layer 33a according to an aspect of the present disclosure may include at least one or more of calcium titanate (CaTiO₃), BaTiO₃, and SrTiO₃, each without lead (Pb), but aspects of the present disclosure are not limited thereto.

The piezoelectric layer 33a according to another aspect of the present disclosure may include a potassium sodium niobite (KNN)-based piezoelectric material including potassium (K), sodium (Na), and niobium (Nb). For example, the KNN-based material may include (K₁₋ₓNaₓ)NbO₃, but aspects of the present disclosure are not limited thereto.

The piezoelectric layer 33a according to another aspect of the present disclosure may include one or more of a first additive and a second additive. The piezoelectric layer 33a may include a KNN-based material including a KCuTaO (KCT)-based additive (or a first additive). An additive may be added for enhancing a mechanical quality factor Qm of the piezoelectric layer 33a. For example, the additive may be added for increasing the mechanical quality factor Qm of the piezoelectric layer 33a to 1,000 or more. For example, the additive may include K_{5.4}C_{1.3}Ta₁₀O₂₉, but aspects of the present disclosure are not limited thereto.

The piezoelectric layer 33a according to another aspect of the present disclosure may include a KNN-based material including a hardener (or a second additive). For example, because the piezoelectric layer 33a is an ultrasound region where a driving range is very high, the piezoelectric layer 33a may include a material having the mechanical quality factor Qm to decrease heat created by driving the piezoelectric layer 33a. The hardener may be added for reducing heat of the piezoelectric layer 33a and increasing the mechanical quality factor Qm of the piezoelectric layer 33a to 1,000 or more. For example, the hardener may be a co-dopant for increasing the mechanical quality factor Qm to 1,000 or more. For example, the hardener may include one or more of CuO, Fe₂O₃, La₂O₃, ZrO₂, ZnO, SnO₂, CdO, MnO₂, and CuNb₂O₆ or a combination thereof. For example, the piezoelectric layer 33a may have the mechanical quality factor Qm of 1,000 or more.

The piezoelectric layer 33a according to another aspect of the present disclosure may include a KNN-based material to which an additive and a hardener are added together. For example, the piezoelectric layer 33a may include a combination of one or more of the additive and the hardener. For example, the hardener and the additive may include one or more of ZnO+SnO₂, CuO+SnO₂, K₄CuNb₈O₂₃+La₂O₃, MnO₂+K_{5.4}Cu_{1.3}Ta₁₀O₂₉, and CuO+K_{5.4}Cu_{1.3}Ta₁₀O₂₉.

The piezoelectric layer 33a according to another aspect of the present disclosure may include a KNN-based material to which one or more of the additive (or the first additive) and the hardener (or the second additive) are added. For example, the piezoelectric layer 33a may include a first composition of K_{0.5}Na_{0.5}NbO₃−K_{5.4}C_{1.3}Ta₁₀O₂₉+MnO₂ or a second composition of K_{0.5}Na_{0.5}NbO₃−K_{5.4}C_{1.3}Ta₁₀O₂₉+CuO, but aspects of the present disclosure are not limited thereto.

In the piezoelectric layer 33a, a mole ratio (mol %) of the first additive may be 0.1 to 1.5, and a mole ratio (mol %) of the second additive may be 0.1 to 1.0. For example, in the first composition, a mole ratio (mol %) of KCT may be 0.15 to 1.5, and a mole ratio (mol %) of MnO₂ may be 0.1 to 1.0. The piezoelectric layer 33a based on the first composition may have a mechanical quality factor Qm of 1,000 to 1,900. For example, in the second composition, a mole ratio (mol %) of KCT may be 0.1 to 1.5, and a mole ratio (mol %) of CuO may be 0.1 to 1.0. The piezoelectric layer 33a based on the second composition may have a mechanical quality factor Qm of 2,000 to 4,000.

The piezoelectric layer 33a according to an aspect of the present disclosure may have a length (or a diameter) LI which enables generating (or outputting) an ultrasound USW having a frequency of 25 MHz or more. In some aspects, the piezoelectric layer 33a may have the length (or the diameter) LI which enables generating (or outputting) an ultrasound USW having a frequency of 100 MHz to 300 MHz.

The first electrode layer 33b may be disposed at or on a first surface (or an upper surface or a front surface) of the piezoelectric layer 33a. The first electrode layer 33b may have the same size as that of the piezoelectric layer 33a, or may have a size which is smaller than that of the piezoelectric layer 33a.

The second electrode layer 33c may be disposed on a second surface (or a lower surface or a rear surface) that is opposite to or different from the first surface of the piezoelectric layer 33a. The second electrode layer 33c may have the same size as the piezoelectric layer 33a, or may be smaller than the piezoelectric layer 33a.

According to an aspect of the present disclosure, to prevent electrical short circuit between the first electrode layer 33b and the second electrode layer 33c, each of the first electrode layer 33b and the second electrode layer 33c may be formed at the other portion, except a periphery portion, of the piezoelectric layer 33a. For example, the first electrode layer 33b may be formed on an entire first surface other than a periphery portion of the piezoelectric layer 33a. For example, the second electrode layer 33c may be formed on an entire second surface other than a periphery portion of the piezoelectric layer 33a.

One or more of the first electrode layer 33b and the second electrode layer 33c according to an aspect of the present disclosure may be formed of a transparent conductive material, a semitransparent conductive material, or an opaque conductive material. For example, the transparent conductive material or the semitransparent conductive material may include indium tin oxide (ITO) or indium zinc oxide (IZO), but aspects of the present disclosure are not limited thereto. The opaque conductive material may include gold (Au), silver (Ag), platinum (Pt), palladium (Pd), molybdenum (Mo), magnesium (Mg), carbon, or silver (Ag) including glass frit, or the like, or may be made of an alloy thereof, but aspects of the present disclosure are not limited thereto. For example, to enhance an electrical characteristic and/or a vibration characteristic of the piezoelectric layer 33a, each of the first electrode layer 33b and the second electrode layer 33c may include silver (Ag) having a low resistivity. For example, the opaque conductive material may be a carbon-based material. Non-limiting examples of a carbon-based material include a carbon black, ketjen black, carbon nanotube, and a carbon material including graphite, but aspects of the present disclosure are not limited thereto.

The plurality of vibration parts 33 may be respectively disposed on the plurality of first signal lines 31 to have a predetermined separation interval DI. For example, the first electrode layer 33b of each of the plurality of vibration parts 33 may be electrically connected to a corresponding first signal line 31 of the plurality of first signal lines 31.

The plurality of second signal lines 35 may be disposed on the second surface 10b of the base member 10 to respectively overlap the plurality of vibration parts 33. For example, the plurality of second signal lines 35 may be disposed on (or over) the second surface 10b of the base member 10 to respectively overlap the plurality of vibration parts 33. The plurality of second signal lines 35 may be configured at the second surface 10b of the base member 10 to electrically connect each of the plurality of vibration parts 33. The second electrode layer 33c of each of the plurality of vibration parts 33 may be connected to a corresponding second signal line 35 of the plurality of second signal lines 35. For example, the plurality of second signal lines 35 may be disposed on (or over) the second surface 10b of the base member 10 to electrically connect the second electrode layer 33c to the plurality of vibration parts 33. The plurality of second signal lines 35 may be disposed on the second surface 10b of the base member 10 to be electrically disconnected (or insulated) from each of the plurality of first signal lines 31 and electrically connected to the second electrode layer 33c of each of the plurality of vibration parts 33. For example, the plurality of second signal lines 35 may be disposed on (or over) the second surface 10b of the base member 10 to be electrically disconnected (or insulated) from each of the plurality of first signal lines 31 and electrically connected to the second electrode layer 33c of each of the plurality of vibration parts 33. For example, the plurality of second signal lines 35 may be disposed on the second surface 10b of the base member 10 to extend along the second direction Y and to be separated from one another in the first direction X.

The plurality of second signal line 35 may be directly formed at the second surface 10b and connected to each of the plurality of vibration parts 33. For example, the plurality of second signal line 35 may be formed at an entire second surface 10b of the base member 10 by patterning a transparent conductive layer deposited on the entire second surface 10b of the base member 10, but aspects of the present disclosure are not limited thereto. The plurality of second signal lines 35 may be formed by a sputtering process using a mask.

Referring to FIGS. 2 and 3, the plurality of second signal lines 35 may contact each of the base member 10 and the plurality of vibration parts 33. For example, the plurality of second signal lines 35 may directly contact each of the base member 10 and the plurality of vibration parts 33. For example, the plurality of second signal lines 35 may contact respective lateral surfaces and upper surfaces of the plurality of vibration parts 33. Each of the plurality of second signal lines 35 may include a plurality of protrusion portions 35a and a plurality of connection portions 35b.

The plurality of protrusion portions 35a may respectively overlap the plurality of vibration parts 33. The plurality of protrusion portions 35a may respectively contact the lateral surfaces and the second electrode layers 33c of the plurality of vibration parts 33.

Each of the plurality of connection portions 35b may contact or directly contact the second surface 10b of the base member 10 between the plurality of protrusion portions 35a. Each of the plurality of connection portions 35b may be separated from the first electrode layer 33b provided in each of the plurality of vibration parts 33. For example, the first electrode layer 33b may not be disposed at a first surface periphery portion of the piezoelectric layer 33a and each of the plurality of connection portions 35b may be separated from the first electrode layer 33b of a corresponding vibration part 33 of the plurality of vibration parts 33. Accordingly, the plurality of second signal lines 35 may be electrically insulated (e.g., disconnected) from the plurality of first signal lines 31, respectively to prevent an electrical short circuit between the plurality of second signal lines 35 and the plurality of first signal lines 31.

Each of the plurality of first signal lines 31 and the plurality of second signal lines 35 may be configured in or formed of a transparent conductive material. For example, each of the plurality of first signal lines 31 and the plurality of second signal lines 35 may include indium tin oxide (ITO) or indium zinc oxide (IZO), but aspects of the present disclosure are not limited thereto.

Each of the plurality of vibration parts 33 may be disposed or configured at an intersection part between the plurality of first signal lines 31 and the plurality of second signal lines 35. For example, each of the plurality of vibration parts 33 may be disposed or interposed at a corresponding intersection between the first signal line 31 and the second signal line 35. Accordingly, each of the plurality of vibration parts 33 may vibrate based on a driving signal applied through the first signal line 31 and the second signal line 35. In some aspects, the plurality of vibration parts 33 may be configured to generate (or output) an ultrasound USW or may vibrate the base member 10 to output (or generate) the ultrasound USW.

The protection member 50 may be disposed or configured at the second surface 10b of the base member 10 to protect the vibration generating part 30. For example, the protection member 50 may be disposed or configured under the second surface 10b of the base member 10 to protect the vibration generating part 30. For example, the protection member 50 may be disposed or configured to face the second surface 10b of the base member 10 with the vibration generating part 30 therebetween The protection member 50 may also be referred to as an insulation layer, an insulation member, a protection layer, or a protection film, but aspects of the present disclosure are not limited thereto.

The protection member 50 may be connected or coupled to the second surface 10b of the base member 10 by an adhesive layer 40. For example, the protection member 50 may be attached on or coupled to the second surface 10b of the base member 10 and the vibration generating part 30 by a film laminating process using an adhesive layer 40.

The adhesive layer 40 may be disposed or formed at the second surface 10b of the base member 10 to surround the vibration generating part 30. For example, the adhesive layer 40 may be disposed or formed between the second surface 10b of the base member 10 and the protection member 50 to surround the plurality of vibration parts 33. The adhesive layer 40 may be disposed or formed at the second surface 10b of the base member 10 to completely surround the plurality of vibration parts 33, the plurality of first signal lines 31, and the plurality of second signal lines 35. For example, the adhesive layer 40 may be disposed or formed between the second surface 10b of the base member 10 and the protection member 50 to completely surround the plurality of vibration parts 33, the plurality of first signal lines 31, and the plurality of second signal lines 35. The vibration generating part 30 may be surrounded by the second surface 10b of the base member 10 and the adhesive layer 40. For example, the vibration generating part 30 may be completely surrounded by the second surface 10b of the base member 10 and the adhesive layer 40. For example, the vibration generating part 30 may be buried or embedded between the base member 10 and the protection member 50. For example, the adhesive layer 40 may include an electrically insulating material which has adhesiveness and is capable of compression and decompression. For example, the adhesive layer 40 may include a transparent adhesive material such as a pressure sensitive adhesive (PSA), an optically cleared adhesive (OCA), or an optically cleared resin (OCR), an epoxy resin, an acrylic resin, a silicone resin, or a urethane resin, or the like, but aspects of the present disclosure are not limited thereto.

Referring to FIGS. 2 and 3, the vibration apparatus 1 according to an aspect of the present disclosure may further include a pad part 80 and a signal supply member 90.

The pad part 80 may be configured at one periphery portion of the base member 10. For example, the pad part 80 may be provided at a center portion of the one periphery portion of the base member 10.

The pad part 80 may include a plurality of pads which are respectively connected to the plurality of first signal lines 31 and the plurality of second signal lines 35 through a plurality of routing lines 85. Each of the plurality of routing lines 85 may be disposed along a periphery portion of the base member 10 and may be electrically connected to an end of each of the plurality of first signal lines 31 and the plurality of second signal lines 35.

The signal supply member 90 may be configured to receive a driving signal from an ultrasound driving circuit and supply the driving signal to the pad part 80. The signal supply member 90 may include a plurality of signal supply lines that are electrically connected to the plurality of pads of the pad part 80, respectively.

An end portion (e.g., a distal end portion) of the signal supply member 90 may overlap the adhesive layer 40. The end portion (e.g., the distal end portion) of the signal supply member 90 may be disposed or inserted (or accommodated) between a portion of the base member 10 and the protection member 50. For example, the end portion (e.g., the distal end portion) of the signal supply member 90 may be disposed or inserted (or accommodated) into the adhesive layer 40 between a portion of the base member 10 and the protection member 50. Accordingly, the signal supply member 90 may be integrated into the vibration apparatus 1. The end portion (e.g., the distal end portion) of the signal supply member 90 may be disposed or inserted (or accommodated) and fixed into the adhesive layer 40 between a portion of the base member 10 and the protection member 50. A contact defect may be prevented between the pad part 80 and the signal supply member 90 that is caused by the movement of the signal supply member 90. For example, the signal supply member 90 may be configured as a signal cable, a flexible cable, a flexible printed circuit cable, a flexible flat cable, a single-sided flexible printed circuit, a single-sided flexible printed circuit board, a flexible multilayer printed circuit, or a flexible multilayer printed circuit board, but aspects of the present disclosure are not limited thereto.

A manufacturing process for forming the vibration part 33 including the piezoelectric layer 33a according to an aspect of the present disclosure will be described below. In some aspects, the piezoelectric layer 33a may be formed at different times and different processes as further described below.

First, a raw material powder of Na₂CO₃, K₂CO₃, Nb₂O₅, Ta₂O₅, and MnO₂ (or CuO) may be weighed based on a mole ratio of a composition which is to be synthesized and may be put into a suitable container (e.g., a nylon jar), and then, may be mixed with an appropriate amount of solvent. For example, the solvent may be an organic solvent such as ethanol, but aspects of the present disclosure are not limited thereto. A step of weighing a raw material may be performed regardless of a manufacturing method, or may be omitted.

Subsequently, a primary ball milling process may be performed on a mixed powder. The primary ball milling process includes putting the mixed power into the solvent and mixing and milling the mixed powder for 24 hours, but aspects of the present disclosure are not limited thereto.

Subsequently, the manufacturing method may separate a ball from the mixed powder, which is milled by the primary ball milling process, and may put the ball into a container and may dry the ball at a temperature of 100° C.

Subsequently, the manufacturing method may grind a dried mixed powder with a mortar into a grinded mixed powder and put the grinded mixed powder into an alumina crucible. The manufacturing method includes increasing a temperature of the grinded mixed powder in an electric furnace at a temperature increasing speed of 5° C./min, calcining the mixed powder for 6 hours at 850° C., and cooling the calcined mixed powder at a room temperature, thereby manufacturing a phase-synthesized matrix material.

In some aspects, a secondary ball milling process may be performed on the phase-synthesized matrix material. The secondary ball milling process comprises putting a matrix material into a solvent and mixing and milling the matrix material for 24 hours to mill the matrix material into a small-particle powder, but aspects of the present disclosure are not limited thereto. For example, the secondary ball milling process may include placing the phase-synthesized matrix material or the powder and a solvent into a container (e.g., a nylon jar) and mixing and milling the solvent-containing matrix material for 24 hours to decrease a size of a particle along with MnO₂ (or CuO), but aspects of the present disclosure are not limited thereto.

Subsequently, the manufacturing method may comprise separating a ball from the matrix material powder, which is milled by the secondary ball milling process, and placing the ball into a container (e.g., a dish) and may dry the ball at a temperature of 100° ° C. Aspects of the present disclosure are not limited to the dry temperature and the dry time.

In some cases, the manufacturing method may include sieving out the dried powder by using a 40-mesh sieve to produce a powder including particles having a certain size or less (e.g., filtering the powder based on size). For example, a powder passing through the 40-mesh sieve may have a size of 400 μm or less, but aspects of the present disclosure are not limited thereto.

Subsequently, the manufacturing method may include molding (e.g., using a press mold) the sieved powder into manufacture a matrix pressing element. For example, the sieved powder may be placed into a mold having a circular shape and may be press-molded, but aspects of the present disclosure are not limited thereto. In a non-limiting example, the pressure in the press molding comprises 100 kg/f(force), but aspects of the present disclosure are not limited thereto.

Subsequently, the manufacturing method may include placing the matrix pressing element into an electric furnace, increasing a temperature of the matrix pressing element at a temperature increasing speed of 5° C./min, sintering the matrix pressing element for 3 to 6 hours within a range of 1,070° ° C. to 1,110° C., and cooling (e.g., using ambient air) the sintered matrix pressing element at a room temperature, thereby manufacturing a sinter or the piezoelectric layer 33a. Aspects of the present disclosure are not limited to the sintering speed and the sintering temperature.

Subsequently, the electrode layers 33b and 33c may be formed in the sinter or the piezoelectric layer 33a. For example, the first electrode layer 33b may be coated on a first surface of the piezoelectric layer 33a or the sinter and dried, and the second electrode layer 33c may be coated on a second surface of the piezoelectric layer 33a or the sinter and dried. For example, the first electrode layer 33b and the second electrode layer 33c may include metal, for example, silver (Ag) paste, but aspects of the present disclosure are not limited thereto. The electrode layers 33b and 33c may be coated on the piezoelectric layer 33a or the sinter and a temperature thereof may increase at a speed of 5° C./min and may be maintained for 10 minutes at a temperature of 600° C., and then, the electrode layers 33b and 33c may be naturally cooled at a room temperature, but aspects of the present disclosure are not limited thereto.

Subsequently, polarization may be formed in the piezoelectric layer 33a or the sinter where the electrode layers 33b and 33c are formed using, for example, a polarization (or poling) process. In one aspect, the polarization process may apply an oil (for example, silicon oil) to the piezoelectric layer 33a or the sinter and may apply an electrical field of 3 kV/mm for a period of time (e.g., 20 minutes) to form or align polarization in the piezoelectric layer 33a.

The vibration apparatus 1 according to an aspect of the present disclosure may generate (or output) an ultrasound USW having a frequency of 25 MHz or more based on a vibration of each of the plurality of vibration parts 33. In this case, the vibration apparatus 1 may generate the ultrasound USW for sterilizing or disinfecting a screen of an apparatus. For example, the disinfecting may remove various bacteria and viruses from a surface of a screen of the apparatus. Therefore, the vibration apparatus 1 according to an aspect of the present disclosure may be used as or applied to an apparatus for sterilizing or disinfecting a screen of the apparatus from harmful bacteria, such as various bacteria and/or viruses of a surface, by using the ultrasound USW. For example, the ultrasound USW having a frequency of 25 MHZ or more may vibrate viruses attached on the surface of the screen may damage a spike protein or a skin (or a cell wall) of the virus, thereby sterilizing the virus. For example, the ultrasound USW having a frequency of 25 MHz or more may vibrate air of the surface of the screen, and thus, may sterilize viruses by using air as a medium or may remove foreign materials, such as dust or fine dust of the surface of the screen, from the surface of the screen.

FIG. 4 illustrates a vibration apparatus according to another aspect of the present disclosure. FIG. 4 is a cross-sectional view taken along line I-I′ illustrated in FIG. 1. FIG. 4 illustrates an aspect where an insulation layer is additionally provided in the vibration generating part of the vibration apparatus according to an aspect of the present disclosure described above with reference to FIGS. 1 to 3. In the following description, an insulation layer of the vibration generating part and relevant elements will be only described, the other elements may be referred to by same reference numerals as FIGS. 1 to 3, and their repetitive descriptions are omitted.

Referring to FIGS. 1 and 4, in the vibration apparatus 1 according to another aspect of the present disclosure, a vibration generating part 30 may include a plurality of first signal lines 31, a plurality of vibration parts 33, an insulation layer 34, and a plurality of second signal lines 35.

Each of the plurality of first signal lines 31 and the plurality of vibration parts 33 may be the same as or substantially the same as the plurality of first signal lines 31 and the plurality of vibration parts 33 as described above with reference to FIGS. 1 to 3, and thus, repeated descriptions thereof are omitted.

The insulation layer 34 may be formed between the plurality of vibration parts 33. The insulation layer 34 may be configured to electrically insulate a first electrode layer 33b of each of the plurality of vibration parts 33 from each other, and each of the plurality of first signal lines 31 from each other. The insulation layer 34 may be configured to fix a position of each of the plurality of vibration parts 33.

The insulation layer 34 may include a transparent insulating material for electrically insulating each of the plurality of first signal lines 31 and the first electrode layer 33b of each of the plurality of vibration parts 33. For example, the insulation layer 34 may include a transparent inorganic material or a transparent organic material or a combination thereof, or may be formed in a stack structure thereto, but aspects of the present disclosure are not limited thereto. For example, the insulation layer 34 may be a transparent insulating layer, an inorganic insulating layer, an organic insulating layer, or a planarization layer, but aspects of the present disclosure are not limited thereto.

The insulation layer 34 may be disposed on a second surface of the base member 10 to surround lateral surfaces of each of the plurality of vibration parts 33. The insulation layer 34 may be provided to surround the lateral surfaces of the vibration parts 33 and allow the second electrode layer 33c to contact the second signal lines 35. The insulation layer 34 may be disposed or filled between the plurality of vibration parts 33, and thus, may fix a position of each of the plurality of vibration parts 33 to be electrically connected to the plurality of first signal lines 31. Accordingly, an electrical connection state (or a contact state) between each of the plurality of first signal lines 31 and the first electrode layer 33b of each of the plurality of vibration parts 33 may be maintained.

According to an aspect of the present disclosure, the insulation layer 34 may be configured to surround a lateral surface of the piezoelectric layer 33a in each of the plurality of vibration parts 33. For example, a height of the insulation layer 34 may be equal to that of the piezoelectric layer 33a. For example, with respect to a thickness direction Z of the base member 10, a distance (or a height) between the second surface 10b of the base member 10 and a surface of the insulation layer 34 may be equal to a distance (or a height) between the second surface 10b of the base member 10 and a surface of the piezoelectric layer 33a.

According to another aspect of the present disclosure, the insulation layer 34 may be configured to surround at least a portion of a lateral surface of the second electrode layer 33c and the lateral surfaces of the piezoelectric layer 33a provided in each of the plurality of vibration parts 33. For example, a height of the insulation layer 34 may be at least equal to a height of the second electrode layer 33c or may be higher than that of the piezoelectric layer 33a and may be lower than that of the second electrode layer 33c.

The plurality of second signal lines 35 may be disposed on the insulation layer 34. For example, the insulation layer 34 may be configured to surround the lateral surfaces of each of the plurality of second signal lines 35. For example, the plurality of second signal lines 35 may be configured at the insulation layer 34 to respectively overlap the plurality of vibration parts 33 (e.g., from the perspective of a plan view). The plurality of second signal lines 35 may be configured at the insulation layer 34 to electrically connect to the second electrode layer 33c of each of the plurality of vibration parts 33. The plurality of second signal lines 35 may be disconnected (or insulated) from each of the plurality of first signal lines 31 by the insulation layer 34 and electrically connected to the second electrode layer 33c of each of the plurality of vibration parts 33. For example, the plurality of second signal lines 35 may extend along the second direction Y and may be configured at the insulation layer 34 to be separated from one another in the first direction X.

The plurality of second signal lines 35 may be formed below the insulation layer 34 to electrically connect to the second electrode layer 33c of each of the plurality of vibration parts 33. For example, the plurality of second signal lines 35 may be directly formed below the insulation layer 34 to electrically connect to the second electrode layer 33c of each of the plurality of vibration parts 33. The plurality of second signal lines 35 may directly contact the insulation layer 34 and the second electrode layer 33c of each of the plurality of vibration parts 33. The plurality of second signal lines 35 may be formed by patterning a transparent conductive layer, which is deposited (or formed) on an entire surface of the insulation layer 34, but aspects of the present disclosure are not limited thereto. For example, the plurality of second signal lines 35 may be formed by patterning a transparent conductive layer which is deposited (or formed) on (or over) an entire surface of the insulation layer 34, but aspects of the present disclosure are not limited thereto. For example, the plurality of second signal lines 35 may be directly formed at the insulation layer 34 by a sputtering process using a mask. For example, the plurality of second signal lines 35 may be directly formed on (or over) the insulation layer 34 by a sputtering process using a mask.

The protection member 50 may be disposed or configured at the second surface 10b of the base member 10 to protect the vibration generating part 30. For example, the protection member 50) may be disposed or configured under the second surface 10b of the base member 10 to protect the vibration generating part 30. The protection member 50 may be disposed or configured at the second surface 10b of the base member 10) to cover the vibration generating part 30. For example, the protection member 50 may be disposed or configured to face the second surface 10b of the base member 10 with the vibration generating part 30 therebetween.

The protection member 50 may be connected or coupled to the second surface 10b of the base member 10 by an adhesive layer 40. For example, the protection member 50 may be attached on or coupled to the second surface 10b of the base member 10 and the vibration generating part 30 by a film laminating process using the adhesive layer 40.

The adhesive layer 40 may be formed at the second surface 10b of the base member 10 to surround the vibration generating part 30. For example, the adhesive layer 40 may be disposed or formed between the second surface 10b of the base member 10 and the protection member 50) to surround the plurality of vibration parts 33. For example, the adhesive layer 40 may be disposed or formed at the second surface 10b of the base member 10 to wholly surround the insulation layer 34, a portion of the second electrode layer 33c of the plurality of vibration parts 33, the plurality of first signal lines 31, and the plurality of second signal lines 35. For example, the adhesive layer 40 may be disposed or formed on (or over) the second surface 10b of the base member 10 to completely surround the insulation layer 34, a portion of the second electrode layer 33c of the plurality of vibration parts 33, the plurality of first signal lines 31, and the plurality of second signal lines 35. For example, the adhesive layer 40 may include an epoxy resin, an acrylic resin, a silicone resin, or a urethane resin, or the like, but aspects of the present disclosure are not limited thereto.

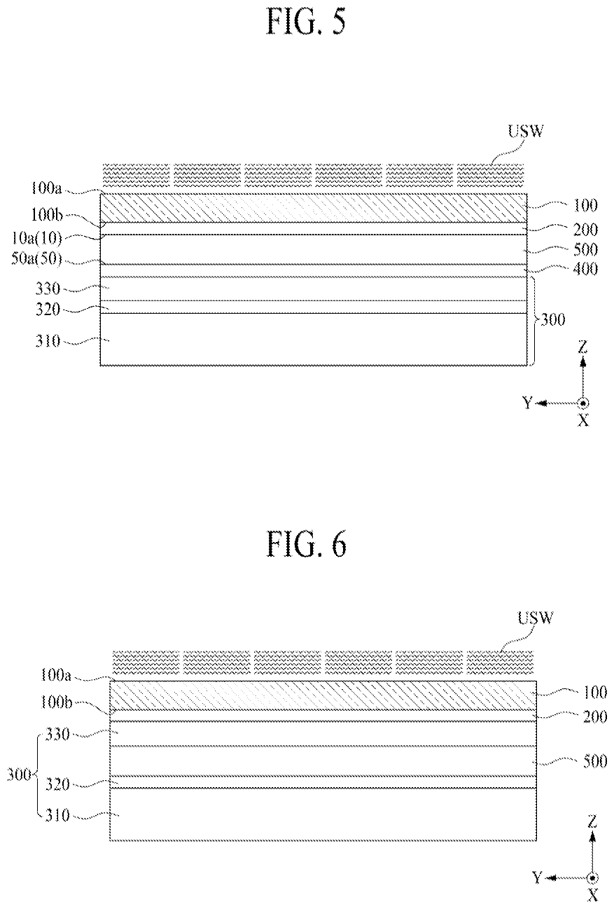

FIG. 5 illustrates an apparatus according to an aspect of the present disclosure.

Referring to FIG. 5, the apparatus according to an aspect of the present disclosure may be an electronic device or a display apparatus including a touch screen. For example, the apparatus according to an aspect of the present disclosure may be an electronic device with a touch screen integrated therein or a display apparatus with a touch screen integrated therein. For example, the apparatus according to an aspect of the present disclosure may be a mobile electronic device such as a mobile phone, a smart phone, a smart watch, tablet PC (personal computer) or a watch phone, or the like, a smart television, an electronic whiteboard, a bidirectional information transfer transparent display, a bidirectional digital signage, a notebook computer, a monitor, a refrigerator, a kiosk, or an automated teller machine, or the like, but aspects of the present disclosure are not limited thereto. For example, the apparatus according to an aspect of the present disclosure may be applied to all electric devices including a touch screen.

The apparatus according to an aspect of the present disclosure may include a front member 100 and a vibration generating apparatus 500.

The front member 100 may provide an exterior surface of the apparatus and may protect a screen of a display apparatus 300. The front member 100 may be disposed at a front surface of the display apparatus 300. For example, the front member 100 may cover a front surface of the display apparatus 300, and thus, may protect the display apparatus 300 from an external impact.

The front member 100 according to an aspect of the present disclosure may include a transparent plastic material, a glass material, or a reinforced glass material, but aspects of the present disclosure are not limited thereto. For example, the front member 100 may include a front structure, a front window, a cover window, a glass window, a cover screen, a screen cover, a window glass or the like, but aspects of the present disclosure are not limited thereto.

The vibration generating apparatus 500 may be disposed between the front member 100 and the display apparatus 300. The vibration generating apparatus 500 may be configured to vibrate the front member 100. The vibration generating apparatus 500 may be configured to generate an ultrasound USW at a front surface 100a (or a surface) of the front member 100. The vibration generating apparatus 500 may vibrate the front member 100 to generate the ultrasound USW at the front surface 100a of the front member 100. The vibration generating apparatus 500) may sterilize or disinfect harmful bacteria such as various bacteria and/or viruses of the front surface 100a of the front member 100 with the ultrasound USW. For example, the vibration generating apparatus 500 may be referred to as a sound wave generating apparatus, an ultrasound generating apparatus, an ultrasound generating device, or an ultrasound vibration apparatus, or the like, but aspects of the present disclosure are not limited thereto. For example, the vibration generating apparatus 500) may be provided to have a size which is the same as or different from that of the front member 100, but aspects of the present disclosure are not limited thereto. For example, the vibration generating apparatus 500 may be disposed to have a size which is smaller than that of the front member 100. For example, the vibration generating apparatus 500 may be disposed at a center part of the front member 100.

According to another aspect of the present disclosure, a member may be further disposed between the front member 100 and the vibration generating apparatus 500. For example, the member may be between a first coupling member 200 and the vibration generating apparatus 500. For example, the member may include one or more of stainless steel, aluminum (Al), a magnesium (Mg), an Al alloy, a Mg alloy, and a magnesium-lithium (Mg—Li) alloy, but aspects of the present disclosure are not limited thereto. For example, when the member is added, the stiffness of the vibration generating apparatus 500 may increase.

The ultrasound USW may have a frequency of 25 MHz or more. For example, the ultrasound USW may have a frequency of 100 MHz to 350 MHZ, but aspects of the present disclosure are not limited thereto. For example, the ultrasound USW having a frequency of 25 MHz or more may vibrate air of the front member 100, and thus, may sterilize harmful bacteria such as various bacteria and/or viruses that are attached on the front surface 100a of the front member 100 by using air as a medium or may remove foreign materials such as dust or fine dust. In some aspects, based on a vibration of the front member 100, a spike protein or a skin (or a cell wall) of virus that attached on the front surface 100a of the front member 100 may be damaged, thereby sterilizing the virus.

The vibration generating apparatus 500 may be provided close to the front member 100, to generate the ultrasound USW at the front surface 100a (or the surface) of the front member 100.

The vibration generating apparatus 500 according to an aspect of the present disclosure may include the vibration apparatus 1 described above with reference to FIGS. 1 to 4. The descriptions of the vibration apparatus 1 described above with reference to FIGS. 1 to 4 may be included in descriptions of the vibration generating apparatus 500 illustrated in FIG. 5, and thus, repeated descriptions thereof are omitted.

The apparatus according to an aspect of the present disclosure may further include a display apparatus 300.

The display apparatus 300 may be disposed at a rear surface of the front member 100. The display apparatus 300 may be configured at a rear surface of the vibration generating apparatus 500. The display apparatus 300 may be configured to display an image. The image may include an electronic image, a digital image, a still image, or a video image or the like. The display apparatus 300 may include a display panel 310 and a touch panel 330.

The display panel 310 may display an image by outputting light. The display panel 310 may be a curved display panel, or may be any type of display panel, such as a liquid crystal display panel, an organic light emitting display panel, a quantum dot light emitting display panel, a micro light emitting diode display panel, and an electrophoresis display panel, or the like. The display panel 310 may be a flexible display panel. For example, the display panel 310 may a flexible light emitting display panel, a flexible electrophoretic display panel, a flexible electro-wetting display panel, a flexible micro light emitting diode display panel, or a flexible quantum dot light emitting display panel, but aspects of the present disclosure are not limited thereto.

When the display panel 310 is the liquid crystal display panel, the display panel 310 may include a plurality of gate lines, a plurality of data lines, and a plurality of pixels respectively provided in the intersection area of the gate lines and the data lines. In addition, the display panel 310 may include a first substrate including a thin film transistor (TFT) which is a switching element for adjusting a light transmittance of each of the plurality of pixels, a second substrate including a color filter and/or a black matrix or the like, and a liquid crystal layer provided between the first substrate and the second substrate.

When the display panel 310 is the organic light emitting display panel, the display panel 310 may include a plurality of gate lines, a plurality of data lines, and a plurality of pixels respectively provided at the intersection area of the gate lines and the data lines. In addition, the display panel 310 may include a substrate including a TFT for selectively applying a voltage to each of the plurality of pixels, an organic light emitting device layer on the substrate, and an encapsulation layer (or an encapsulation substrate) disposed at the substrate to cover the organic light emitting device layer, or the like. The encapsulation substrate may protect the TFT and the organic light emitting device layer or the like from an external impact and may prevent water or oxygen from penetrating into the organic light emitting device layer. Moreover, the light emitting device layer may be configured as an inorganic light emitting layer (for example, a nano-sized material layer) and/or a quantum dot light emitting layer, or the like. As another aspect of the present disclosure, the organic light emitting device layer may be changed to a micro light emitting diode.

The touch panel 330 may be disposed between the display panel 310 and the front member 100. The touch panel 330 may be configured to sense a user touch applied to the front member 100. For example, the touch panel 330 may be configured to sense the user touch based on a finger or a touch pen, but aspects of the present disclosure are not limited thereto.

The touch panel 330 may include a touch electrode layer for sensing a finger touch or a pen touch of a user applied to the front member 100. The touch electrode layer may be configured to sense a capacitance variation at a touch electrode based on the user's touch. For example, the touch electrode layer may include an electrode structure corresponding to a mutual-capacitance type and is configured in a manner that a plurality of touch driving electrodes and a plurality of touch sensing electrodes cross each other or a self-capacitance type configured with only a plurality of touch sensing electrodes, but aspects of the present disclosure are not limited thereto.

The touch panel 330 according to an aspect of the present disclosure may be disposed at or connected to a front surface of the display panel 310 by an adhesive member 320. The adhesive member 320 may be a transparent adhesive member or a first adhesive member, but aspects of the present disclosure are not limited thereto. For example, the adhesive member 320 may include a transparent adhesive material such as an OCA, an OCR, or a PSA, or the like, but aspects of the present disclosure are not limited thereto.

The touch panel 330) according to another aspect of the present disclosure may be replaced with an in-cell touch panel configured inside the display panel 310, and in this case, the adhesive member 320 may be omitted. For example, when the display panel 310 is a liquid crystal display panel, the touch panel 330 may use a common electrode of the display panel 310 as a touch electrode to sense the user touch. In another example, when the display panel 310 is an organic light emitting display panel, the touch panel 330 may be disposed on the encapsulation layer of the display panel 310, but aspects of the present disclosure are not limited thereto. For example, the in-cell touch panel may be a touch electrode layer or a touch sensor layer, but aspects of the present disclosure are not limited thereto.

The vibration generating apparatus 500 according to an aspect of the present disclosure may generate an ultrasound USW periodically or at every predetermined period, but aspects of the present disclosure are not limited thereto. For example, the vibration generating apparatus 500 may generate the ultrasound USW when a user touch occurs, or may generate the ultrasound USW for a predetermined time after the user touch.

The vibration generating apparatus 500 according to another aspect of the present disclosure may be configured to, when a user touch occurs, generate the ultrasound USW and provide a tactile feedback to the user. The ultrasound USW output from the vibration generating apparatus 500) may be realized a squeeze film effect and may provide the tactile feedback to a finger of the user. The squeeze film effect may be referred to as surface ultrasonic lubrication, and the surface ultrasonic lubrication may change a friction coefficient (or a frictional force) between the finger of the user and the front member 100 through changing of a friction coefficient of a surface of the front member 100 to implement fine texture or roughness recognizable by the user. For example, the ultrasound USW for the tactile feedback may have a frequency of 20 KHz or more, but aspects of the present disclosure are not limited thereto.

The vibration generating apparatus 500 may be coupled to or attached on a rear surface 100b (or a backside surface) of the front member 100 by a first coupling member 200. The first coupling member 200 may be configured or interposed between the front member 100 and the display apparatus 300. For example, the first coupling member 200 may be configured between the base member 10 of the vibration generating apparatus 500) and the front member 100. For example, the first coupling member 200 may be configured between a first surface 10a of the base member 10 of the vibration generating apparatus 500) and a rear surface 100b of the front member 100.

The first coupling member 200 may include a transparent adhesive material such as a PSA, an OCA, an OCR, an epoxy resin, an acrylic resin, a silicone resin, or a urethane resin, or the like, but aspects of the present disclosure are not limited thereto. For example, the first coupling member 200 may include the acrylic-based adhesive member which is relatively better in adhesive force and has high hardness so that a vibration of the vibration generating apparatus 500 may be well transferred to the front member 100.

The vibration generating apparatus 500 may be coupled to or attached on a front surface of the display apparatus 300 by a second coupling member 400. The second coupling member 400 may be configured or interposed between the vibration generating apparatus 500) and the touch panel 330 of the display apparatus 300. For example, the second coupling member 400 may be configured between the protection member 50 of the vibration generating apparatus 500 and the touch panel 330. For example, the second coupling member 400 may be configured between a rear surface 50a of the protection member 50 of the vibration generating apparatus 500) and a front surface of the touch panel 330.

The second coupling member 400 may include a transparent adhesive material such as a PSA, an OCA, or an OCR, an epoxy resin, an acrylic resin, a silicone resin, or a urethane resin, or the like, but aspects of the present disclosure are not limited thereto. The second coupling member 400 may be configured as a same or different adhesive material as the first coupling member 200. For example, the second coupling member 400 may be configured to minimize or prevent the transfer of a vibration of the front member 100 to the display apparatus 300. The second coupling member 400 may include a material characteristic suitable for blocking a vibration. For example, the second coupling member 400 may include a material having elasticity to absorb vibration (e.g., impact absorption). For example, the second coupling member 400 may include a urethane-based material having a ductile characteristic.

Based on a vibration of the vibration generating apparatus 500 according to an aspect of the present disclosure, the ultrasound USW may be generated at the front surface 100a of the front member 100. The vibration generating apparatus 500 may sterilize harmful bacteria such as various bacteria and/or viruses attached on the front surface 100a of the front member 100 and may displace (e.g., remove) foreign materials such as dust or fine dust. Also, the apparatus according to an aspect of the present disclosure may change a frictional coefficient (or a frictional force) between the finger of the user and the front member 100 by using the ultrasound USW when a user touch occurs, and thus, may provide a tactile feedback to the user.

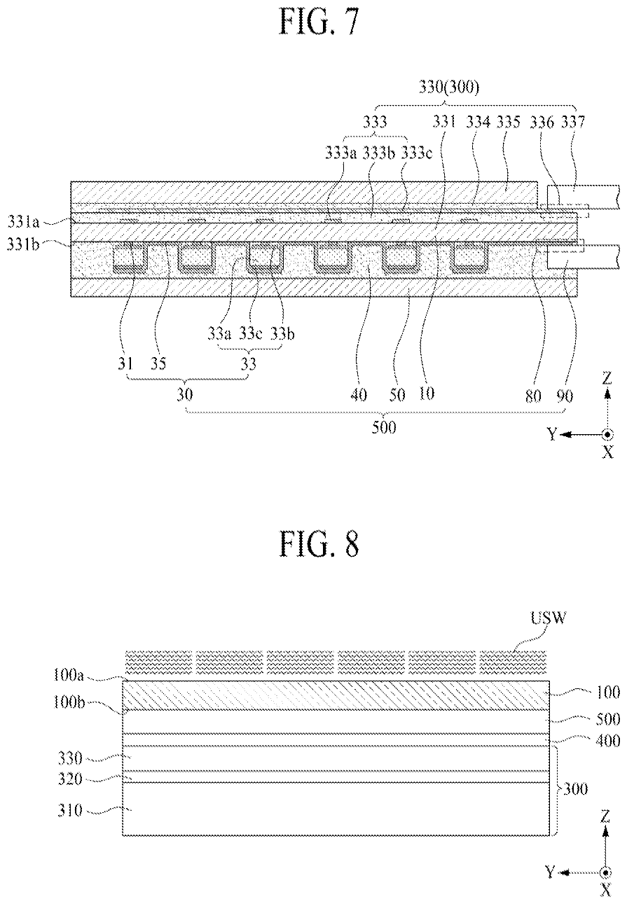

FIG. 6 illustrates an apparatus according to another aspect of the present disclosure. FIG. 7 illustrates a vibration generating apparatus and a touch panel illustrated in FIG. 6. FIGS. 6 and 7 illustrate an aspect where a vibration apparatus is configured at a touch panel of a display apparatus in an apparatus according to an aspect of the present disclosure. In the following description, therefore, the touch panel and the vibration apparatus and relevant elements will be only described, the other elements may be referred to by same reference numerals as FIG. 5, and their repetitive descriptions are omitted.

Referring to FIGS. 6 and 7, in an apparatus according to another aspect of the present disclosure, a touch panel 330) of a display apparatus 300 may be configured to include a vibration generating apparatus 500. The touch panel 330 may vibrate based on a vibration of the vibration generating apparatus 500 to vibrate the front member 100. The touch panel 330 may transfer a vibration of the vibration generating apparatus 500 to the front member 100 to vibrate the front member 100.

The touch panel 330 according to an aspect of the present disclosure may include a first substrate 331, a touch electrode layer 333, and a second substrate 335.

The first substrate 331 may include a first surface 331a (or a front surface) adjacent to the front member 100 and a second surface 331b (or a rear surface) adjacent to the display panel 310. The first substrate 331 may be a lower substrate, a first base film, or a lower base film, or the like, but aspects of the present disclosure are not limited thereto. For example, the first substrate 331 may include a transparent plastic material, a glass material, or a reinforced glass material, but aspects of the present disclosure are not limited thereto.

The touch electrode layer 333 may be disposed between the first substrate 331 and the second substrate 335. The touch electrode layer 333 may be disposed on the first surface 331a of the first substrate 331. For example, the touch electrode layer 333 may be disposed on an inner surface of the second substrate 335.

The touch electrode layer 333 according to an aspect of the present disclosure may include a plurality of first touch electrodes 333a disposed at the second surface 331b of the first substrate 331, a touch insulation layer 333b covering the plurality of first touch electrodes 333a, and a plurality of second touch electrodes 333c intersecting with each of the plurality of first touch electrodes 333a. For example, the touch electrode layer 333 may sense a user touch based on a change in capacitance in an intersection region between the first touch electrode 333a and the second touch electrode 333c.

The second substrate 335 may be configured to cover the touch electrode layer 333. The second substrate 335 may be coupled to or attached on the first surface 331a of the first substrate 331 and the touch electrode layer 333 by an adhesive layer 334. The second substrate 335 may be an upper substrate, a second base film, or an upper base film, or the like, but aspects of the present disclosure are not limited thereto. For example, the second substrate 335 may include a transparent plastic material, a glass material, or a reinforced glass material, but aspects of the present disclosure are not limited thereto.

The adhesive layer 334 may include a transparent adhesive material such as a PSA, an OCA, or an OCR, an epoxy resin, an acrylic resin, a silicone resin, or a urethane resin, or the like, but aspects of the present disclosure are not limited thereto. For example, the adhesive layer 334 may include the acrylic-based adhesive member having a relatively higher adhesive force and hardness so that a vibration of the vibration generating apparatus 500 may be well transferred to the front member 100.

The touch panel 330 may be coupled or connected to the rear surface 100b of the front member 100 by a first coupling member 200. For example, the second substrate 335 of the touch panel 330 may be coupled or connected to the rear surface 100b of the front member 100 by a first coupling member 200.

The touch panel 330 according to an aspect of the present disclosure may further include a touch pad part 336 and a touch signal supply member 337.

The touch pad part 336 may include a plurality of touch pads disposed at one periphery portion of the first substrate 331. The plurality of touch pads may be respectively connected to the plurality of first touch electrodes 333a and the plurality of second touch electrodes 333c through touch routing lines in a one-to-one connection relationship, but aspects of the present disclosure are not limited thereto.

The touch signal supply member 337 may be provided to be electrically connected to the touch pad part 336. Therefore, a host controller (or a touch controller) of the apparatus may be configured to sense a capacitance variation of the touch electrode layer 333 through the touch signal supply member 337 and the plurality of touch routing lines. For example, the host controller (or the touch controller) may supply a touch driving signal to each of the plurality of first touch electrodes 333a through the touch signal supply member 337 and the plurality of touch routing lines and may be configured to sense a capacitance variation in an intersection region between the first touch electrode 333a and the second touch electrode 333c through each of the plurality of second touch electrodes 333c.

The vibration generating apparatus 500 may be configured to vibrate the touch panel 330, and thus, may generate an ultrasound USW at a surface of the front member 100 by a vibration of the front member 100 based on a vibration of the touch panel 330. The vibration generating apparatus 500 may be configured between the touch panel 330 and the display panel 310. The vibration generating apparatus 500 may be integrated or configured at the touch panel 330. For example, the vibration generating apparatus 500 may be configured or directly formed at the first substrate 331 of the touch panel 330. For example, the vibration generating apparatus 500 may be configured or directly formed at the second surface 331b of the first substrate 331.

The vibration generating apparatus 500 may include a base member 10, a vibration generating part 30, and a protection member 50.

The base member 10 may be the first substrate 331 or the second substrate 335 of the touch panel 330. For example, the first substrate 331 of the touch panel 330 may be used as the base member 10 of the vibration generating apparatus 500.

The vibration generating part 30 may include a plurality of first signal lines 31, a plurality of vibration parts 33, and a plurality of second signal lines 35, which are configured to contact the second surface 331b of the first substrate 331. For example, the vibration generating part 30 may include a plurality of first signal lines 31, a plurality of vibration parts 33, and a plurality of second signal lines 35, which are configured on the second surface 331b of the first substrate 331. The vibration generating part 30 may include a plurality of first signal lines 31 and a plurality of second signal lines 35, which are arranged on the base member 10 to intersect with one another at the second surface 331b of the first substrate 331, and a plurality of vibration parts 33 which are disposed or interposed between the plurality of first signal lines 31 and the plurality of second signal lines 35 in intersection portions (or intersection regions) between the plurality of first signal lines 31 and the plurality of second signal lines 35. For example, the vibration generating part 30 may include a plurality of first signal lines 31 and a plurality of second signal lines 35, which are configured to intersect with one another under the second surface 331b of the first substrate 331, and a plurality of vibration parts 33 which are disposed or interposed between the plurality of first signal lines 31 and the plurality of second signal lines 35 at intersection portions (or intersection regions) between the plurality of first signal lines 31 and the plurality of second signal lines 35.

Except for that the plurality of first signal lines 31, the plurality of vibration parts 33, and the plurality of second signal lines 35 are provided on the second surface 331b of the first substrate 331, the plurality of first signal lines 31, the plurality of vibration parts 33, and the plurality of second signal lines 35 may be the same as or substantially the same as the plurality of first signal lines 31, the plurality of vibration parts 33, and the plurality of second signal lines 35 described above with reference to FIGS. 1 to 4, respectively, and thus, repeated descriptions thereof are omitted.

The protection member 50 may be disposed or configured at the second surface 331b of the first substrate 331 to protect the vibration generating part 30. For example, the protection member 50 may be disposed or configured under the second surface 331b of the first substrate 331 to protect the vibration generating part 30. The protection member 50 may be disposed or configured at the second surface 331b of the first substrate 331 to cover the vibration generating part 30. For example, the protection member 50 may be disposed or configured under the second surface 331b of the first substrate 331 to cover the vibration generating part 30 therebetween. For example, the protection member 50 may be an insulating layer, an insulating member, a protective layer, or a protective film, or the like, but aspects of the present disclosure are not limited thereto.

The protection member 50 may be connected or coupled to the second surface 331b of the first substrate 331 by an adhesive layer 40. For example, the protection member 50 may be attached on or coupled to the second surface 331b of the first substrate 331 and the vibration generating part 30 by a film laminating process using the adhesive layer 40.

The vibration generating apparatus 500 may be coupled to or connected to a front surface of the display panel 310 by an adhesive member 320. For example, the adhesive member 320) may be configured or interposed between a protection member 50 of the vibration generating apparatus 500 and the front surface of the display panel 310.

The adhesive layer 40 and the protection member 50 of the vibration generating apparatus 500 may be omitted. In this case, the adhesive member 320 may be provided or interposed between the display panel 310 and the base member 10) (or the first substrate 331 of the touch panel 330) of the vibration generating apparatus 500 to surround the vibration generating part 30 of the vibration generating apparatus 500, and thus, may couple or connect the vibration generating apparatus 500 to the display panel 310.

According to another aspect of the present disclosure, the ultrasound USW may be generated at the front surface 100a of the front member 100 based on a vibration of the vibration generating apparatus 500. In this case, the vibration generating apparatus 500) may sterilize harmful bacteria such as various bacteria and/or viruses attached on the front surface 100a of the front member 100 or may displace (e.g., remove) foreign materials such as dust or fine dust. In addition, the apparatus according to another aspect of the present disclosure may change a frictional coefficient (or a frictional force) between the finger of the user and the front member 100 by using the ultrasound USW when a user touch occurs, and thus, may provide a tactile feedback to the user. Because the vibration generating apparatus 500 and the touch panel 330 are provided as a single body, the apparatus according to another aspect of the present disclosure may have a thin thickness and the first substrate 331 of the touch panel 330 or the base member of the vibration generating apparatus 500 may be omitted, and thus, the number of elements may be reduced and the manufacturing cost may decrease.

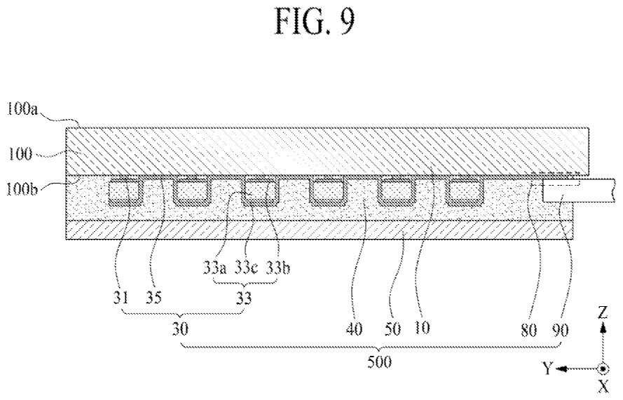

FIG. 8 illustrates an apparatus according to another aspect of the present disclosure. FIG. 9 illustrates a front member and a vibration apparatus illustrated in FIG. 8. FIGS. 8 and 9) illustrate an aspect where a vibration generating apparatus is configured as a front member in an apparatus. In the following description, the vibration apparatus and relevant elements will be only described, the other elements may be referred to by same reference numerals as FIG. 5, and their repetitive descriptions are omitted.

Referring to FIGS. 8 and 9, in an apparatus according to another aspect of the present disclosure, the vibration generating apparatus 500 may be disposed or configured between the front member 100 and the display apparatus 300 (or a touch panel 330). The vibration generating apparatus 500 may be integrated or configured at the front member 100. For example, the vibration generating apparatus 500 may be configured or directly formed on the front member 100. For example, the vibration generating apparatus 500 may be configured or directly formed at a rear surface 100b of the front member 100. The vibration generating apparatus 500 may be configured to generate an ultrasound USW at a surface of the front member 100. For example, the vibration generating apparatus 500 may be configured to directly vibrate the front member 100, and thus, may generate an ultrasound USW at the surface of the front member 100 based on a vibration of the front member 100. In this case, a first coupling member 200 between the front member 100 and the vibration generating apparatus 500 described above with reference to FIG. 5 may be omitted.

The vibration generating apparatus 500 may include a base member 10, a vibration generating part 30, and a protection member 50.

The base member 10 may be a front member 100. For example, the front member 100 may be used as the base member 10 of the vibration generating apparatus 500.

The vibration generating part 30 may include a plurality of first signal lines 31, a plurality of vibration parts 33, and a plurality of second signal lines 35, which are configured at a rear surface 100b of the front member 100. For example, the vibration generating part 30 may include a plurality of first signal lines 31 and a plurality of second signal lines 35, which are configured on the rear surface 100b of the front member 100 to intersect with one another, and a plurality of vibration parts 33 which are disposed or interposed between the plurality of first signal lines 31 and the plurality of second signal lines 35 in intersection portions (or intersection regions) between the plurality of first signal lines 31 and the plurality of second signal lines 35.

Except for that each of the plurality of first signal lines 31, the plurality of vibration parts 33, and the plurality of second signal lines 35 are provided on the rear surface 100b of the front member 100. The plurality of first signal lines 31, the plurality of vibration parts 33, and the plurality of second signal lines 35 may be the same as or substantially the same as the plurality of first signal lines 31, the plurality of vibration parts 33, and the plurality of second signal lines 35 described above with reference to FIGS. 1 to 4, respectively, and thus, repeated descriptions thereof are omitted.

The protection member 50 may be disposed or configured at the rear surface 100b of the front member 100 to protect the vibration generating part 30. For example, the protection member 50 may be disposed or configured at the rear surface 100b of the front member 100 to cover the vibration generating part 30. For example, the protection member 50 may be an insulating layer, an insulating member, a protective layer, or a protective film, or the like, but aspects of the present disclosure are not limited thereto.

The protection member 50 may be connected or coupled to the rear surface 100b of the front member 100 by an adhesive layer 40. For example, the protection member 50 may be attached on or coupled to the rear surface 100b of the front member 100 by a film laminating process using the adhesive layer 40.

The vibration generating apparatus 500 may be coupled to or attached to a front surface of the display apparatus 300 by a second coupling member 400. For example, the second coupling member 400 may be configured or interposed between a protection member 50 of the vibration generating apparatus 500 and a front surface of the display apparatus 300. For example, the second coupling member 400 may be configured or interposed between the protection member 50 of the vibration generating apparatus 500 and a front surface of a touch panel 330 of the display apparatus 300.

According to another aspect of the present disclosure, the adhesive layer 40 and the protection member 50 of the vibration generating apparatus 500 may be omitted. For example, the second coupling member 400 may be configured or interposed between the display apparatus 300 and the base member 10 (or the rear surface of the front member 100) of the vibration generating apparatus 500 to surround the vibration generating part 30 of the vibration generating apparatus 500. In this case, the second coupling member 400 may couple or connect the vibration generating apparatus 500 to the display apparatus 300. For example, when the adhesive layer 40 and the protection member 50 of the vibration generating apparatus 500 are omitted, the second coupling member 400 may be configured or interposed between the touch panel 330 of the display apparatus 300 and the base member 10 (or the rear surface of the front member 100) of the vibration generating apparatus 500 to surround the vibration generating part 30 of the vibration generating apparatus 500, and thus, may couple or connect the vibration generating apparatus 500 to the touch panel 330 of the display apparatus 300.

Based on a vibration of the vibration generating apparatus 500 according to another aspect of the present disclosure, the ultrasound USW may be generated at the front surface 100a of the front member 100, and thus, may sterilize harmful bacteria such as various bacteria and/or viruses attached on the front surface 100a of the front member 100 or may remove foreign materials such as dust or fine dust. In addition, the apparatus according to another aspect of the present disclosure may change a frictional coefficient (or a frictional force) between the finger of the user and the front member 100 by using the ultrasound USW when a user touch occurs, and thus, may provide a tactile feedback to the user. Because the vibration generating apparatus 500 and the touch panel 330 are provided as one body, the apparatus according to another aspect of the present disclosure may have a thin thickness and the base member of the vibration generating apparatus 500 may be omitted, and thus, the number of elements may be reduced and the manufacturing cost may decrease.

A vibration apparatus and an apparatus including the same according to an aspect of the present disclosure are described below.

A vibration apparatus according to an aspect of the present disclosure may comprise a base member, a protection member, and a vibration generating part configured between the base member and the protection member to output an ultrasound, the vibration generating part may comprise a plurality of first signal lines and a plurality of second signal lines configured on the base member to intersect with one another, and a plurality of vibration parts disposed at intersections the plurality of first signal lines and the plurality of second signal lines.

According to one or more aspects of the present disclosure, each of the plurality of first signal lines may contact the base member and a first surface of a portion of the plurality of vibration parts. Each of the plurality of second signal lines may contact the base member and at least a second surface of a different portion of the plurality of vibration parts.

According to one or more aspects of the present disclosure, each of the plurality of second signal lines may contact a lateral surface and an upper surface of each of the different portion of the plurality of vibration parts.

According to one or more aspects of the present disclosure, the vibration generating part may further comprise an insulation layer between the plurality of vibration parts. Each of the plurality of second signal lines may be disposed on the insulation layer.

According to one or more aspects of the present disclosure, the insulation layer may be configured to surround a lateral surface of each of the plurality of second signal lines.

According to one or more aspects of the present disclosure, the ultrasound may comprise a frequency of 25 MHz or more.

According to one or more aspects of the present disclosure, a separation interval between the plurality of vibration parts may be 0.5 cm to 8 cm.

According to one or more aspects of the present disclosure, the separation interval may be configured based on a minimum distance associated with a destructive interference between the plurality of vibration parts and a maximum distance associated with an ultrasonic transmission distance of each of the plurality of vibration parts.

According to one or more aspects of the present disclosure, each of the plurality of vibration parts may comprise a piezoelectric layer, a first electrode layer at a first surface of the piezoelectric layer, and a second electrode layer at a second surface different from the first surface of the piezoelectric layer. The first electrode layer of each of the plurality of vibration parts may be connected to a corresponding first signal line of the plurality of first signal lines. The second electrode layer of each of the plurality of vibration parts may be connected to a corresponding second signal line of the plurality of second signal lines.

According to one or more aspects of the present disclosure, the piezoelectric layer may have a mechanical quality factor of 1,000 or more.

According to one or more aspects of the present disclosure, the piezoelectric layer may be configured as a lead (Pb)-free piezoelectric material.

According to one or more aspects of the present disclosure, the piezoelectric layer may comprise a potassium sodium niobite (KNN)-based piezoelectric material including potassium (K), sodium (Na), and niobium (Nb).

According to one or more aspects of the present disclosure, the piezoelectric layer may comprise at least one of a first additive and a second additive. The first additive may comprise a KCuTaO-based material. The second additive may comprise one or more of CuO, Fe₂O₃, La₂O₃, ZrO₂, ZnO, SnO₂, CdO, MnO₂, and CuNb₂O₆ or a combination thereof.

According to one or more aspects of the present disclosure, a mole ratio of the first additive may be 0.1 to 1.5. A mole ratio of the second additive may be 0.1 to 1.0.

According to one or more aspects of the present disclosure, each of the first electrode layer and the second electrode layer may have a size same as or smaller than that of the piezoelectric layer.

According to one or more aspects of the present disclosure, each of the first electrode layer and the second electrode layer may be formed at the other portion, except a periphery portion, of the piezoelectric layer.

According to one or more aspects of the present disclosure, wherein each of the plurality of second signal lines may include a plurality of protrusion portions and a plurality of connection portions. The plurality of protrusion portions respectively may overlap the plurality of vibration parts. Each of the plurality of connection portions may contact a surface of the base member between the plurality of protrusion portions.

According to one or more aspects of the present disclosure, each of the plurality of connection portions may be spaced apart from the first electrode layer of a corresponding vibration part of the plurality of vibration parts.

According to one or more aspects of the present disclosure, the vibration apparatus may further comprise a signal supply member. An end portion of the signal supply member may be inserted between a portion of the base member and the protection member

According to one or more aspects of the present disclosure, the vibration apparatus may further comprise an adhesive layer between the base member and the protection member. The end portion of the signal supply member may be inserted and fixed into the adhesive layer between a portion of the base member and the protection member.

According to one or more aspects of the present disclosure, the vibration generating part may further comprise an insulation layer between the plurality of vibration parts. The insulation layer may be configured to electrically insulate a first electrode layer of each of the plurality of vibration parts from each other, and each of the plurality of first signal lines from each other.

According to one or more aspects of the present disclosure, the vibration generating part may further comprise a connector coupled to the plurality of first signal lines and the plurality of second signal lines for activating the plurality of vibration parts and an adhesive layer disposed on the plurality of second signal lines for fixing the connector into a fixed position and planarizing a layer.

According to one or more aspects of the present disclosure, the plurality of vibration parts may be configured to output the ultrasound signal to damage a microscopic surface or protein of material accumulated on the base member.

An apparatus according to an aspect of the present disclosure may comprise a front member, a vibration generating apparatus configured to generate an ultrasound at a surface of the front member. The vibration generating apparatus may comprise a vibration apparatus. The vibration apparatus may comprise a base member, a protection member, and a vibration generating part configured between the base member and the protection member to output an ultrasound. The vibration generating part may comprise a plurality of first signal lines and a plurality of second signal lines configured on the base member to intersect with one another, and a plurality of vibration parts disposed at intersection of the plurality of first signal lines and the plurality of second signal lines.

According to one or more aspects of the present disclosure, each of the plurality of vibration parts of the vibration apparatus may comprise a piezoelectric layer, a first electrode layer at a first surface of the piezoelectric layer, and a second electrode layer at a second surface different from the first surface of the piezoelectric layer. The first electrode layer of each of the plurality of vibration parts may be connected to a corresponding first signal line of the plurality of first signal lines. The second electrode layer of each of the plurality of vibration parts may be connected to a corresponding second signal line of the plurality of second signal lines.

An apparatus according to an aspect of the present disclosure may comprise a front member, a display apparatus at a rear surface of the front member, and a vibration generating apparatus between the front member and the display apparatus and configured to vibrate the front member and generate an ultrasound at a surface of the front member. The vibration generating apparatus may comprise a vibration apparatus. The vibration apparatus may comprise a base member, a protection member, and a vibration generating part configured between the base member and the protection member to output an ultrasound. The vibration generating part may comprise a plurality of first signal lines and a plurality of second signal lines configured on the base member to intersect with one another, and a plurality of vibration parts disposed at intersections of the plurality of first signal lines and the plurality of second signal lines.

According to one or more aspects of the present disclosure, each of the plurality of vibration parts of the vibration apparatus may comprise a piezoelectric layer, a first electrode layer at a first surface of the piezoelectric layer, and a second electrode layer at a second surface different from the first surface of the piezoelectric layer. The first electrode layer of each of the plurality of vibration parts may be connected to a corresponding first signal line of the plurality of first signal lines. The second electrode layer of each of the plurality of vibration parts may be connected to a corresponding second signal line of the plurality of second signal lines.

According to one or more aspects of the present disclosure, the display apparatus may comprise a touch panel and a display panel. The vibration generating apparatus may between the front member and the touch panel, or may be between the touch panel and the display panel.

According to one or more aspects of the present disclosure, the display apparatus may comprise a display panel and a touch panel between the display panel and the front member. The touch panel may comprise a first substrate, a second substrate, and a touch electrode layer between the first substrate and the second substrate. The base member of the vibration generating apparatus may be the first substrate or the second substrate of the touch panel.

According to one or more aspects of the present disclosure, the base member of the vibration generating apparatus may be the front member.

A method of forming a vibration generating layer for cresting acoustic vibrations according to one or more aspects of the present disclosure may comprise forming a plurality of first signal lines on a substrate in a first direction, attaching a plurality of vibration parts to the plurality of first signal lines, forming a plurality of second signal lines over the plurality of vibration parts in a second direction, and depositing an adhesive over the plurality of first signal lines, the plurality of vibration parts, and the plurality of second signal lines.

According to one or more aspects of the present disclosure, the plurality of vibration parts may be configured to generate an ultrasound signal to damage a microscopic surface or protein of material accumulated on an exterior surface of a device.

A vibration apparatus or a transparent vibration apparatus according to one or more aspects of the present disclosure may be applied to or included in a vibration apparatus disposed at a display apparatus. The display apparatus according to one or more aspects of the present disclosure may be applied to or included in mobile apparatuses, video phones, smart watches, watch phones, wearable apparatuses, foldable apparatuses, rollable apparatuses, bendable apparatuses, flexible apparatuses, curved apparatuses, sliding apparatuses, variable apparatuses, electronic organizers, electronic books, portable multimedia players (PMPs), personal digital assistants (PDAs), MP3 players, mobile medical devices, desktop personal computers (PCs), laptop PCs, netbook computers, workstations, navigation apparatuses, automotive navigation apparatuses, automotive display apparatuses, automotive apparatuses, theatre apparatuses, theatre display apparatuses, TVs, wall paper display apparatuses, signage apparatuses, game machines, notebook computers, monitors, cameras, camcorders, and home appliances, or the like. In addition, the vibration apparatus or the transparent vibration apparatus according to one or more aspects of the present disclosure may be applied to or included in an organic light-emitting lighting apparatus or an inorganic light-emitting lighting apparatus. When the vibration apparatus or the transparent vibration apparatus is applied to or included in the lighting apparatuses, the lighting apparatuses may act as lighting and a speaker. In addition, when the vibration apparatus or the transparent vibration apparatus according to one or more aspects of the present disclosure is applied to or included in the mobile apparatuses, or the like, the vibration apparatus or the transparent vibration apparatus may be one or more of a speaker, a receiver, and a haptic device, but aspects of the present disclosure are not limited thereto.

It will be apparent to those skilled in the art that various modifications and variations may be made in the present disclosure without departing from the scope of the disclosure. Thus, it is intended that the present disclosure covers the modifications and variations of this disclosure provided that within the scope of the claims and their equivalents.

What is claimed is:

1. A vibration apparatus, comprising:
a base member;
a protection member; and
a vibration generating part disposed between the base member and the protection member to output an ultrasound signal,
wherein the vibration generating part comprises:
a plurality of first signal lines and a plurality of second signal lines configured on the base member to intersect with one another; and
a plurality of vibration parts disposed at intersections of the plurality of first signal lines and the plurality of second signal lines.

2. The vibration apparatus of claim 1, wherein each of the plurality of first signal lines contacts the base member and a first surface of a portion of the plurality of vibration parts, and
wherein each of the plurality of second signal lines contacts the base member and at least a second surface of a different portion of the plurality of vibration parts.

3. The vibration apparatus of claim 2, wherein each of the plurality of second signal lines contacts a lateral surface and an upper surface of each of the different portion of the plurality of vibration parts.

4. The vibration apparatus of claim 1, wherein the vibration generating part further comprises an insulation layer disposed between the plurality of vibration parts; and
wherein the plurality of second signal lines are formed on the insulation layer.

5. The vibration apparatus of claim 4, wherein the insulation layer is configured to surround a lateral surface of each of the plurality of second signal lines.

6. The vibration apparatus of claim 1, wherein the ultrasound signal comprises a frequency of at least 25 MHz or more.

7. The vibration apparatus of claim 1, wherein a separation interval between each of the plurality of vibration parts is 0.5 cm to 8 cm.

8. The vibration apparatus of claim 7, wherein the separation interval is configured based on a minimum distance associated with a destructive interference between the plurality of vibration parts and a maximum distance associated with an ultrasonic transmission distance of each of the plurality of vibration parts.

9. The vibration apparatus of claim 1, wherein each of the plurality of vibration parts comprises:
a piezoelectric layer;
a first electrode layer at a first surface of the piezoelectric layer; and
a second electrode layer at a second surface different from the first surface of the piezoelectric layer, and
wherein the first electrode layer of each of the plurality of vibration parts is connected to a corresponding first signal line of the plurality of first signal lines,
wherein the second electrode layer of each of the plurality of vibration parts is connected to a corresponding second signal line of the plurality of second signal lines.

10. The vibration apparatus of claim 9, wherein the piezoelectric layer has a mechanical quality factor of at least 1,000.

11. The vibration apparatus of claim 9, wherein the piezoelectric layer comprises a lead (Pb)-free piezoelectric material.

12. The vibration apparatus of claim 9, wherein the piezoelectric layer comprises a potassium sodium niobite (KNN)-based piezoelectric material including potassium (K), sodium (Na), and niobium (Nb).

13. The vibration apparatus of claim 12, wherein the piezoelectric layer comprises at least one of a first additive and a second additive:
wherein the first additive comprises a KCuTaO-based material; and
wherein the second additive comprises one or more of CuO, Fe₂O₃, La₂O₃, ZrO₂, ZnO, SnO₂, CdO, MnO₂, and CuNb₂O₆ or a combination thereof.

14. The vibration apparatus of claim 13, wherein a mole ratio of the first additive is 0.1 to 1.5 and a mole ratio of the second additive is 0.1 to 1.0.

15. The vibration apparatus of claim 9, wherein each of the first electrode layer and the second electrode layer has a size same as or smaller than that of the piezoelectric layer.

16. The vibration apparatus of claim 9, wherein each of the first electrode layer and the second electrode layer is formed at the other portion, except a periphery portion, of the piezoelectric layer.

17. The vibration apparatus of claim 9, wherein each of the plurality of second signal lines includes a plurality of protrusion portions and a plurality of connection portions,
wherein the plurality of protrusion portions respectively overlap the plurality of vibration parts, and
wherein each of the plurality of connection portions contacts a surface of the base member between the plurality of protrusion portions.

18. The vibration apparatus of claim 17, wherein the plurality of protrusion portions respectively contact lateral surfaces and the second electrode layers of the plurality of vibration parts.

19. The vibration apparatus of claim 17, wherein each of the plurality of connection portions is spaced apart from the first electrode layer of a corresponding vibration part of the plurality of vibration parts.

20. The vibration apparatus of claim 9, wherein the vibration generating part further comprises an insulation layer between the plurality of vibration parts, and
wherein the insulation layer is configured to electrically insulate a first electrode layer of each of the plurality of vibration parts from each other, and each of the plurality of first signal lines from each other.

21. The vibration apparatus of claim 1, further comprising a signal supply member,
wherein an end portion of the signal supply member is inserted between a portion of the base member and the protection member.

22. The vibration apparatus of claim 21, further comprising an adhesive layer between the base member and the protection member,
wherein the end portion of the signal supply member is inserted and fixed into the adhesive layer between a portion of the base member and the protection member.

23. The vibration apparatus of claim 1, further comprising:
a connector coupled to the plurality of first signal lines and the plurality of second signal lines for activating the plurality of vibration parts; and
an adhesive layer disposed on the plurality of second signal lines for fixing the connector into a fixed position and planarizing a layer.

24. The vibration apparatus of claim 1, wherein the plurality of vibration parts are configured to output the ultrasound signal to damage a microscopic surface or protein of material accumulated on the base member.

25. An apparatus, comprising:
a front member;
a vibration generating apparatus configured to generate an ultrasound at a surface of the front member,
wherein the vibration generating apparatus comprises:
a base member;
a protection member; and
a vibration generating part disposed between the base member and the protection member to output an ultrasound signal, wherein the vibration generating part comprises:
a plurality of first signal lines and a plurality of second signal lines configured on the base member to intersect with one another; and
a plurality of vibration parts disposed at intersections of the plurality of first signal lines and the plurality of second signal lines.

26. The apparatus of claim 25, wherein each of the plurality of vibration parts of the vibration generating apparatus comprises:
a piezoelectric layer;
a first electrode layer at a first surface of the piezoelectric layer; and
a second electrode layer at a second surface different from the first surface of the piezoelectric layer, and
wherein the first electrode layer of each of the plurality of vibration parts is connected to a corresponding first signal line of the plurality of first signal lines, and
wherein the second electrode layer of each of the plurality of vibration parts is connected to a corresponding second signal line of the plurality of second signal lines.

27. An apparatus, comprising:
a front member;
a display apparatus at a rear surface of the front member; and
a vibration generating apparatus between the front member and the display apparatus and configured to vibrate the front member,
wherein the vibration generating apparatus comprises:
a base member;
a protection member; and
a vibration generating part configured between the base member and the protection member to output an ultrasound, wherein the vibration generating part comprises:
a plurality of first signal lines and a plurality of second signal lines configured on the base member to intersect with one another; and
a plurality of vibration parts disposed at intersection parts between the plurality of first signal lines and the plurality of second signal lines.

28. The apparatus of claim 27, wherein each of the plurality of vibration parts of the vibration generating apparatus comprises:
a piezoelectric layer;
a first electrode layer at a first surface of the piezoelectric layer; and
a second electrode layer at a second surface different from the first surface of the piezoelectric layer, and
wherein the first electrode layer of each of the plurality of vibration parts is connected to a corresponding first signal line of the plurality of first signal lines, and
wherein the second electrode layer of each of the plurality of vibration parts is connected to a corresponding second signal line of the plurality of second signal lines.

29. The apparatus of claim 27, wherein the display apparatus comprises a touch panel and a display panel, and
wherein the vibration generating apparatus is between the front member and the touch panel, or is between the touch panel and the display panel.

30. The apparatus of claim 27, wherein the display apparatus comprises a display panel and a touch panel between the display panel and the front member,
wherein the touch panel comprises:
a first substrate;
a second substrate; and
a touch electrode layer between the first substrate and the second substrate,
wherein the base member of the vibration generating apparatus is the first substrate or the second substrate of the touch panel.

31. The apparatus of claim 27, wherein the base member of the vibration generating apparatus is the front member.
